(19) United States
(12) Patent Application Publication
(10) Pub. No.: US 2003/0064385 A1
(43) Pub. Date: Apr. 3, 2003

(54) Genes expressed in breast cancer as prognostic and therapeutic targets

(76) Inventors: Marlene Michelle Dressman, Germantown, MD (US); Christian Nicolas Lavedan, Potomac, MD (US); Mihael Polymeropoulos, Potomac, MD (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/120,583

(22) Filed: Apr. 11, 2002

Related U.S. Application Data
(60) Provisional application No. 60/291,428, filed on May 16, 2001.

(51) Int. Cl. C12Q 1/68

(52) U.S. Cl. 435/6; 702/20

(57) ABSTRACT

Methods are disclosed for, determining the endocrine responsiveness of breast carcinoma and treating and monitoring the progression of breast carcinoma based on genes which are differentially expressed in breast tumors. Also disclosed are methods for identifying agents useful in the treatment of breast carcinoma, methods for monitoring the efficacy of a treatment for breast carcinoma, methods for inhibiting the proliferation of a breast carcinoma, and breast-specific vectors including the promoters of the disclosed genes.

This application claims priority to U.S. Provisional Application No. 60/291,428, filed May 16, 2001, which is incorporated by reference herein in its entirety.

### BACKGROUND OF THE INVENTION

### FIELD OF THE INVENTION

This invention relates to methods for the monitoring, prognosis and treatment of cancer. In particular, the invention relates to the use of gene expression analysis to determine endocrine therapy responsiveness of breast cancer and to help choose or monitor the efficacy of various treatments for breast cancer.

### DESCRIPTION OF THE RELATED ART

Breast cancer is the most common cancer affecting American women. In the United States alone, nearly 200,000 new cases of breast cancer are diagnosed each year and some 44,000 women will die of the disease. Breast cancer will occur in 12.5% (1 out of every 8 women) during their lifetimes and account for 32% of cases of cancer in women. It is the second leading cause of female cancer death after lung cancer. Male breast cancer accounts for about 1% of all new cases and has a similar natural history as that in females. Although the incidence of breast cancer is now slowly decreasing, the mortality rate has remained constant for the past several decades. Worldwide, almost 1 million new cases of breast cancer are diagnosed yearly. In general, more affluent Western nations have the highest incidence rates, whereas developing nations have the lowest.

The causes of breast cancer are still unknown, but numerous risk factors have been identified. For example, the incidence of breast cancer increases dramatically with advancing age; more than 50% of women with breast cancer in the United States are older than 60 years. Other risk factors are younger age at menarche and older age at menopause.

More recently, it has been discovered that mutations in the putative tumor suppressor genes, BRCA-1 and BRCA-2, may account for a large percentage of breast cancers. Women with these mutations often have a positive family history and in 5% of all breast cancer patients, a clear pattern of autosomal dominant inheritance is noted (see Cecil, “Textbook of Medicine”, Goldman and Bennett, Eds., Saunders Co., Philadelphia, Pa.).

The treatment of breast cancer and the ultimate outcome depend on the tumor pathology and the staging of the cancer at the time of treatment. The most commonly used staging system is the TNM system. This system determines the state or stage of the cancer, based on the tumor size, the degree of lymph node involvement and the presence of metastasis (see American Joint Committee on Cancer: AJCC Cancer Staging Handbook, Lippincott-Raven, Philadelphia, Pa. (1998)). The stage of the cancer at the time of detection determines the outcome measured as percent free of recurrence at 10 years. This is the percentage of patients who have not experienced a recurrence of the original cancer in the 10 years after the original tumor is removed by mastectomy or lumpectomy.

The symptoms of breast cancer vary a great deal and depend on the location and size of the primary tumor, and the presence, location and extent of metastases. However the symptoms may include one or more of the following: unilateral or bilateral palpable breast mass, nipple discharge, breast skin changes, breast pain, which may or may not be cyclic in nature, i.e., with menses, bloody or watery nipple discharge, a palpable axillary mass, or other evidence of lymph node involvement.

If the primary tumor has metastasized then symptoms may occur in any organ system in the body. The most common metastatic sites are locoregional, i.e., the chest wall and/or regional lymph nodes (20-40%), bone (60%), lung, i.e., malignant effusion and/or parenchymal lesions (15-25%) and the liver (10-20%). Central nervous system (CNS), spinal cord or other skeletal metastases and leptomeningeal metastases can cause local or diffuse pain, especially back pain, and neurological symptoms or dysfunction including, parathesias, paraplegia, weakness or loss of sensation and hypercalcemia. Seizures, headache, mental status changes or even paralysis or stroke are common with CNS involvement. Liver metastases may cause liver failure with elevated liver function tests, jaundice and/or other evidence of liver dysfunction. Lung involvement can cause difficulty breathing, pneumonia or other respiratory symptoms. While the above symptoms are common in breast cancer with or without metastases since the tumor cells can invade and proliferate in any tissue in the body it is possible for almost symptom complex to occur in patients with breast cancer.

Numerous prognostic factors have been identified in breast cancer patients, including the degree of invasion of the tumor locally, the number of involved axillary lymph nodes and tumor size, and these factors are incorporated in the staging system described above.

However, an important predictive factor in breast cancer is the expression on the surface of the tumor cells of estrogen receptor alpha (ESR1). The estrogen receptor (ER) is a ligand actuated transcription factor that regulates the expression of a variety of genes including growth factors, hormones and oncogenes important for the growth of breast cancer (see Gronemeyer, Ann. Rev. Genetics, Vol. 25, pp. 89-123 (1991); Dickson & Lippman, “The Molecular Basis of Cancer”, Mendelsohn, Ed.; Howley, Israel & Liotta, Eds., pp. 358-384, W. B. Saunders Co., Philadelphia, Pa. (1994)). Expression of the ER plays an important role in the pathogenesis and maintenance of breast cancer. In breast cancer patients about two-thirds of tumors are ESR1-positive (see Lippman et al., Cancer, Vol. 46, pp. 2838-2841 (1980)). Approximately 50% of these ER-positive tumors are estrogen-dependent and respond to endocrine therapy (see Manni et al., Cancer, Vol. 46, pp. 2838-2841 (1980); Jensen, Cancer, Vol. 47, pp. 2319-2326 (1981)). Breast carcinomas occurring in postmenopausal women are often ER-positive (see Iglehart, “Textbook of Surgery”, 14^{th} Ed., Sabiston, Ed., pp. 510-550, W. B. Saunders, Philadelphia, Pa. (1991)). Many of these tumors express significantly more ER than does the normal mammary epithelium (see Ricketts et al., Cancer Res., Vol. 51, pp. 1817-1822 (1991)).

The ESR1 gene spans 140 Kb and is comprised of 8 exons that are spliced to yield a 6.3 Kb on RNA encoding a 595-amino acid protein with a molecular weight of 66 kilodaltons (see Walter et al., Proc. Natl. Acad. Sci. USA, Vol. 82, pp. 7889-7893; and Ponglikitmongkoli et al., EMBO J., Vol. 7, pp. 3385-3388).

Patients whose primary lesions express ESR1 have at least a 5-10% improvement in survival compared to patients whose primary lesions do not express ERs.

In addition, and of great importance, the presence of ESR1 in the primary lesion tends to predict a positive response to adjuvant therapy in the form of endocrine therapy. The purpose of the endocrine therapy is to block the activation of ERs on the tumor cells and thereby decrease or stop the growth and proliferation of tumor cell mass.

Multiple approaches have been used to block the activation of ERs in breast cancer patients. The most widely used agents have been the anti-estrogens such as tamoxifen, which inhibits the action of estrogen at the level of the malignant cell. Tamoxifen works as an anti-estrogen drug, although it has both agonist and antagonist actions at the ER. The drug has traditionally been the first-line of treatment for patients with advanced breast cancer.

However, unfortunately, for patients with advanced ER-positive breast cancer the response rate to tamoxifen is only around 50% (see Clark et al, Semin. Oncol., Vol. 15, No. 2, Suppl. 1, pp. 20-25 (1988)). In many cases where there is no response to tamoxifen, the growth of the tumor has seemingly become independent from control by estrogen and the use of anti-estrogen drugs will not work. Surprisingly, however, about a third of tamoxifen-resistant patients will respond to a reduction in endogenous estrogen levels (see Dombernowsky et al., J. Clin. Oncol., Vol. 16920, pp. 453-461 (1998); and Crump et al., Breast Cancer Res. Treat., Vol. 44, No. 3, pp. 201-210 (1997)). In postmenopausal patients this can be achieved with the selective non-steroidal aromatase inhibitor letrozole (Femara™) (see Dombernowsky et al., supra). Femara is an aromatase inhibitor that works by binding to the enzyme aromatase and inhibiting it from converting adrenal androgens to estrogens.

In addition, other agents that produce their clinical effect by reducing the concentration of estrogen available to the target cell have also been used. These include progestins, such as megestrol and medroxy progesterone acetate, LHRH, androgens and other aromatase inhibitors, such as anastrozole (see Litherland et al, Cancer Treatment Reviews, Vol. 15, pp. 183-194 (1988)).

Therefore, in general, patients whose tumors are positive for ERs are good candidates for endocrine therapy. However, as discussed above, only 30-70% of ESR1-positive malignancies will respond to endocrine therapy, e.g., anti-estrogens or estrogen-deprivation therapies (see Clark et al, Semin. Oncol., Vol. 15, pp. 20-25 (1988); and Lutherland et al., Cancer Treatment Reviews, Vol. 15, pp. 183-194 (1988)). The molecular basis for ESR1-positive malignancies that are resistant to endocrine therapy is not well understood.

Attempts have been made to increase the predictive power of biomarkers for breast cancer endocrine therapy by measuring the expression of the estrogen-regulated gene progesterone receptor (PGR) and trefoil factor 1 (TFF1), also known as PS2. The presence of either one of these proteins indicates the presence of a functional and activated ER and both these proteins are predictive biomarkers for breast cancer endocrine therapy. The use of PGR expression improves the predictive value of ESR1 alone, but 20% of tumors that express both ER and PGR still fail to respond to endocrine therapy in the metastatic setting. Likewise, TFF1 is associated with a good prognosis and predicts a positive response to hormonal therapy, but it has not proved to be sufficient as a predictive biomarker for routine evaluation of breast cancer (see Ribieras et al., Biochem. Biophys. Acta., Vol. F-61-F77, p. 1378 (1998)).

The use of methods such as cytosol-based ligand-binding assays or immunohistochemistry (1HC) to evaluate the presence of ERs in breast cancer tumor cells, and the PGR and TFF1 status is valuable in predicting endocrine therapy responsiveness, but a significant number of patients exhibit primary or acquired resistance to endocrine therapy despite the presence of these proteins and the ability to predict whether a given patients tumor will be responsive to endocrine based therapy remains poor.

The identification of genes with expression patterns similar to ESR1 in breast cancer biopsies provides methods to add to the predictive value of ESR1. Furthermore, the key molecular mechanism involved in breast cancer remains largely unknown. The identification of genes which are regulated by or co-expressed with the ER in breast cancer cells is of great importance to the development of biomarkers for hormone responsiveness in breast cancer, elucidating the molecular mechanisms of breast cancer and the development of new therapeutic targets for treating patients with breast cancer or patients at risk of developing breast cancer.

In addition, currently, the principal manner of identifying the presence of breast cancer is through detection of the presence of dense tumorous tissue. This is accomplished, with varying degrees of success, by direct examination of the outside of the breast or through mammography of other X-ray imaging methods (see Jatoi, Am. J. Surg., Vol. 177, pp. 518-524 (1999)). In order to determine if a particular tumor is ESR1-positive or not it has been necessary to obtain a biopsy specimen of the tumor for IHC analysis. This approach is costly and invasive and exposes the patient to complications such as infection. Less invasive diagnostic assays that could be performed on blood would be very desirable since tumor tissue is not always accessible for profiling.

Therefore, there is a need for more specific and less invasive methods to determine if a patients' tumor is ESR1-positive or not. In addition, there is a great need to provide methods to determine how responsive a particular patients' tumor will be to endocrine-based therapy regardless of the presence or absence of ERs. This would allow the physician to make a more informed decision regarding treatment options and allow a much more accurate prognosis to be given to the patient. In addition there is a need for methods to identify compounds that will improve the response rate of breast cancer tumors to endocrine-based therapy.

### SUMMARY OF THE INVENTION

The present invention, as described herein below, overcomes deficiencies in currently available methods of determining hormone responsiveness of ER-positive breast cancer by identifying a plurality of genes which are regulated by/co-expressed with the ER in human breast cancer cells. The mRNA transcripts and proteins corresponding to these genes have utility, e.g., as surrogate markers of hormone responsiveness and as potential therapeutic targets that are specific for breast cancer.

Furthermore the present invention identifies genes which are differentially expressed in breast carcinoma tumors that are responsive to endocrine-based therapy and those that are not responsive, including treatment with the aromatase inhibitor, letrozole (FEMARA™).

The present invention identifies several genes associated with ESR1 expression that encode secreted proteins, these include: TFF1; trefoil factor 3 (TFF3); serine or cysteine proteinase inhibitor, lade A member 3 (SERPINA3); prolactin-induced protein (PIP), matrix Gla protein (MGP); transforming growth factor-beta type III receptor (TGFRB3); and alpha-2-glycoprotein 1, zinc (AZGP1). These proteins could form the basis for serum-based predictive biomarkers. All genes identified in the various embodiments of this invention are listed, with their Unigene Cluster number, gene symbol and the protein accession number for their expressed proteins, in Table 6.

### DETAILED DESCRIPTION OF THE INVENTION

The present invention relates to the identification of genes, which are regulated by or co-expressed with the ER in breast cancer cells. The expression of ESR1 in primary breast carcinomas identifies a tumor phenotype that is associated with endocrine responsiveness, longer disease-free interval and longer overall survival. A highly statistically significant correlation has been found between the expression of the gene for ESR1 and the expression of 18 other genes in a large sample of breast carcinomas. By virtue of the co-expression of these genes with the ER gene in breast cancer cells, these genes and their expression products can be used in the management, prognosis and treatment of patients at risk for, with, or at risk of, recurrence of breast cancer. These genes are identified in Table 1. The complete sequences of these 18 genes and all other genes disclosed in this application are available using the Unigene Cluster accession numbers shown in Table 6.

Methods of detecting the level of expression of mRNA are well-known in the art and include, but are not limited to, northern blofting, reverse transcription PCR, real time quantitative PCR and other hybridization methods.

A particularly useful method for detecting the level of mRNA transcripts obtained from a plurality of the disclosed genes involves hybridization of labeled mRNA to an ordered array of oligonucleotides. Such a method allows the level of transcription of a plurality of these genes to be determined simultaneously to generate gene expression profiles or patterns. The gene expression profile derived from the sample obtained from the subject can, in another embodiment, be compared with the gene expression profile derived form the sample obtained from the disease-free subject, and thereby determine whether the subject has or is at risk of developing breast cancer.

The strong association between the regulation of the ER gene and the regulation of these 18 genes supports the hypothesis that these genes are co-regulated with the ER gene and therefore are biomarkers for a functional ER transcriptosome. Ten of these genes listed in Table 1 (Gene Nos. 8-17) have already been shown to be associated with the ER gene or directly regulated by estrogen. The first seven genes shown in Table 1 (Gene Nos. 1-7, i.e., sodium channel, non-voltage-gated 1 alpha (SCNN1A); SERPINA3; N-acylsphingosine amidohydrolase (ASAH); lipocalin 1 (LCN1); TGFBR3; glutamate receptor precursor 2 (GRIA2) and cytochrome P450, subfamily IIB (phenobarbital-inducible) CYP2B), have never before been shown to be associated with the expression of the ER in breast carcinoma.

Therefore, this invention provides a plurality of genes that are regulated with the ER in a large sample of breast cancers. Any selection, of at least one, of these genes can be utilized as a surrogate ER marker. In particularly useful embodiments, a plurality of these genes can be selected and their mRNA expression monitored simultaneously to provide expression profiles for use in various aspects.

In a further embodiment. The levels of the gene expression products (proteins) can be monitored in various body fluids, including, but not limited to, blood, plasma, serum, lymph, CSF, cystic fluid, ascites, urine, stool and bile. This expression product level can be used as surrogate markers of the presence of ERs on the tumor cells and can provide indices of endocrine therapy responsiveness of the subjects' tumor.

In addition, expression profiles of one or a plurality of these genes could provide valuable molecular tools for examining the molecular basis of endocrine responsiveness in breast cancer and for evaluating the efficacy of drugs for treating breast cancer. Changes in the expression profile from a baseline profile while the cells are exposed to various modifying conditions, such as contact with a drug or other active molecules can be used as an indication of such effects.

The present invention, in another embodiment, provides the identification of genes that are expressed at different levels in the breast carcinoma tumors that will respond to endocrine therapy as compared to those that will not respond to endocrine therapy. By virtue of the differential expression of these genes, it is possible to utilize these genes and/or their expression products to enhance the certainty of prediction of whether a particular breast tumor in a patient will respond favorably to endocrine therapy. These genes are neuro-oncolgoical ventral antigen 1 (NOVA1), and immunoglobulin heavy, constant, gamma chain three (IGHG3) and are listed in Table 2. The level of expression of the disclosed genes can be detected either by measuring the mRNA corresponding to the gene expression or the protein encoded by the gene. The protein can be measured in any convenient body fluid including, but not limited to, blood, plasma, serum, lymph, CSF, cystic fluid, ascites, urine, stool and bile.

Therefore, this invention provides methods for determining whether cells in a particular breast carcinoma sample will have an endocrine responsive phenotype. The term “endocrine responsive” as used herein, means a breast tumor or carcinoma, the growth or proliferation of which can be slowed or prevented by therapy that results in altered, i.e., increased or decreased, activation of the ER on the tumor cells.

The term “endocrine therapy” as used herein, means any type of therapy that, as a major aspect of it's clinical effect, produces, either directly or indirectly, an increase or decrease in the activation of the ER on the tumor cells. Thus the term endocrine therapy includes, but is not limited to, ER-blocking drugs and drugs that are mixed agonist-antagonists at the ER and treatments that reduce the concentration of endogenous estrogen including, but not limited to, e.g., aromatase inhibitors, progestins and LHRH.

Accordingly, this invention provides a method for screening a subject with breast cancer to determine the likelihood that the subjects' breast tumor will respond to endocrine therapy, methods for the identification of agents that are useful in treating a subject having breast cancer, methods for monitoring the efficacy of certain drug treatments for breast cancer and vectors for specific replication in breast cancer tumor cells.

Definitions of Objective Response Used in the Letrozole (FEMARA™) vs. Tamoxifen Comparison Study

Measurable Disease

1. Complete Response (CR): The disappearance of all known disease, determined by 2 observations not less than 4 weeks apart.

2. Partial Response (PR): A 50% or more decrease in total tumor size of the lesions which have been measured to determine the effect of therapy by 2 observations not less than 4 weeks apart. In addition there can be no appearance of new lesions or progression of any lesion.

3. No Change (NC): A 50% decrease in total tumor size cannot be established nor has a 25% increase in the size of one or more measurable lesions been demonstrated.

4. Progressive Disease (PD): A 25% or more increase in the size of one or more measurable lesions, or the appearance of new lesions.

Clinical Response Assessment

The primary efficacy variable was tumor response, assessed by clinical examination using World Health Organization (WHO) criteria (see, WHO Handbook for Reporting Results of Cancer Treatment). It was defined as the percentage of patients in each treatment group with a CR or PR as determined clinically in the breast by palpation at 4 months. Possible responses were CR, PR, NC, PD or not assessable/not evaluable (NA/NE). Palpable ipsilateral axillary lymph nodal involvement downgraded a clinical CR in tumor. Other factors were also considered such the percentage of patients who underwent breast-conserving surgery (quadrantectomy/lumpectomy) instead of mastectomy. Patients who became inoperable, or who remained inoperable at 4 months, were counted as treatment failures.

Methods Used For the Determination of Genes Co-Regulated With the ESR1 in Breast Cancer

Materials and Methods

Cell Culture

U373 cells (ATCC, Rockville, Md.) were grown in DMEM/F-12 plus 0.03 mg/mL endothelial cell growth supplement (ECGS), 0.1 mg/mL Heparin and 1× Pen/Strep. The cells were grown to approximately 40% confluency and then washed once with media. The cells were then grown for 48 hours with either media or media+PDGF 20 ng/mL. Human vein endothelial cells, HUVEC (ATCC, Rockville, Md.), were grown in F-12 media with 5% FBS, 0.03 mg/mL ECGS, 0.1 mg/mL Heparin and 1× Pen/Strep to approximately 40% confluency and then washed once with media. The cells were grown for 48 hours in ether media or media+VEGF 50 ng/mL. Breast cancer cell line MCF7 (ATCC, Rockville, Md.) was grown in MEM+2 mM L-Glutamine, 0.1 mM NEAA, 1 mM sodium pyruvate, 0.1 mM bovine insulin, 10% BSA to a confluency of 80%. All cell cultures were washed twice with ice cold PBS and then scraped from the dish, pelleted in cold PBS and snap frozen in liquid nitrogen.

Sample Preparation

Twenty-one RNA samples were extracted from 14-gauge needle core biopsies collected before initiation of neoadjuvant endocrine therapy from patients enrolled in a randomized Phase III trial of letrozole (FEMARA™, Novartis Pharma, Basal Switzerland) versus tamoxifen for postmenopausal women with primary invasive breast cancer ineligible for breast conserving surgery. RNA was extracted from an additional 30 primary breast adenocarcinomas collected in Sweden, one additional ESR1+breast tumor surgical biopsy, two HUVEC samples, two samples from glioblastoma cell line U373-MG and one MCF7 sample using Trizol (Life Technologies, Gaithersburg, Md.). The clinical samples were collected after informed consent had been obtained according to protocols approved by local ethics committees. RNA was purchased for two samples, an infiltrating Stage III duct carcinoma (Ambion, Austin, Tex.) and a pool of two normal breast tissues (Clontech, Palo Alto, Calif.). The total number of samples prepared was 59 including 53 breast cancer biopsies and one pooled normal breast sample. Total RNA was purified using QIAGEN RNEASY™ columns (Qiagen, Valencia, Calif.), processed and hybridized to the HUGENE™ FL 6800 Array (Affymetrix, Santa Clara, Calif.), as described by Lockhart et al., Nat. Biotechnol., Vol. 14, pp. 1675-1680 (1996).

Hierarchical Clustering

A 1,156-gene subset of the HuGeneFL 6800 array was used as input for clustering due to computational limitations. This subset was comprised of those genes called present by GENECHIP® Software (Affymetrix, Santa Clara, Calif.) in at least one of the 59 samples and that had a 20-fold difference in expression, i.e., average difference (AvDif) between the normal pooled breast tissue sample and at least one of the 59 samples. This subset of genes ideally represented those genes that had some level of variation between normal and tumors. It excluded those genes that were either not expressed in any sample or did not vary significantly in at least one sample. Gene expression values were used to cluster genes and samples using GENESPRING™ 3.2.8 (Silicon Genetics, Redwood City, Calif.), with the average difference measurement for each gene normalized across samples to a median of one. Gene expression similarity was measured by standard correlation with a minimum distance of 0.001 and a separation ratio of 0.5. A list of genes co-clustering with ESR1 was compiled from the branch of the resulting dendogram containing the ESR1 gene.

Results

Experimental Sample Tree

The samples with no or very low ESR1 expression primarily clustered near one end of the dendogram and the samples with high ESR1 expression clustered at the other end despite no clear branch delineating the two sample classes (FIG. 2). The AvDif values for ESR1 ranged from −24.08 to 3501.6 with normal breast exhibiting a value of 124. The normal breast sample clustered at the border of the samples that generally had low expression for the 18 genes reported here and those samples with high expression. The mean of the ESR1 AvDif for all samples clustered above normal breast in FIG. 2 were 66.37 with a standard deviation of 163.54. The mean of the ESR1 AvDif for all samples clustered below the normal breast sample were 1440 with a standard deviation of 936.

Endothelial and glioblastoma cell culture samples clustered with their respective cell types in branches distinct from the tumor biopsies. The endothelial and glioblastoma branches were located at the end of the dendogram with low ESR1 expression. Cell lines were included in the clustering analysis to improve the clustering of genes by providing cell types that may be present in breast tumors, such as endothelial and epithelial, as well as cell types that would clearly be different, such as glioblastoma.

Genes Co-Clustering With ESR1

Eighteen genes co-clustered with ESR1 (Table 1). These genes had a distinct pattern of high expression in the ESR1-positive samples and low expression in the ESR1-negative samples (FIG. 2). Seven of the genes that co-clustered with ESR1 had not previously been associated with estrogen stimulation or breast cancer, i.e., SCNN1A, SERPINA3, ASAH, LCN1, TGFBR3, GRIA2 and CYP2B (Table 1).

Six of the genes co-clustering with ESR1 have previously been considered to be estrogen-regulated proteins, predictive or prognostic biomarkers for breast cancer, i.e., carcinoembryonic antigen-related cell adhesion molecule 5 (CEACAM5), LIV-1 protein (LIV-1), PIP, MGP, TFF3 and TFF1, also known as PS2 (see Table 1).

CEACAM5 is an immunoreactive glycoprotein that is reportedly expressed in 10-95% of breast cancers. CEACAM5 protein level was found to be highest in ESR1-positive/PGR-positive tumors in a study of 298 mammary tissue samples (see Molina et al., Anticancer Res., Vol. 19, pp. 2557-2562 (1999)). In addition to correlating with ESR1 expression, CEACAM5 was found to correlate with mammaglobin 1 (MGB1) expression in a report by Zach et al., J. Clin Oncol, Vol. 17, pp. 2015-2019 (1999). This same report also found that MGB1 levels correlated with ER levels, supporting the gene-clustering results.

LIV-1 is a well-documented ER gene. It is induced by epidermal growth factor (EGF), transforming growth factor alpha (TGFα) and insulin growth factor 1 (IGF1) through an ESR1-dependent mechanism (see El-Tanani et al, J. Steroid Biochem. Mol. Biol., Vol. 60, pp. 269-276 (1997)).

PIP, alternatively known as gross cystic disease fluid protein 15, is induced by prolactin and androgen. PIP expression levels are correlated with ESR1- and PGR-positive status (see Clark et al., Br. J. Cancer, Vol. 81, pp. 1002-1008 (1999)).

MGP belongs to the osteocalcin/matrix gla-protein family that associates with the organic matrix of bone and cartilage and is thought to act as an inhibitor of bone formation. Estrogen is a strong inducer of MGP gene expression.

Estrogen also strongly induces TTF1 and TTF3. Trefoil factors are stable secretory proteins expressed in gastrointestinal mucosa. They may function to protect the mucosal epithelium from insults and aid healing. TFF3 may be a predictive biomarker for breast cancer endocrine therapies. It is expressed in estrogen-responsive but not in estrogen-non-responsive breast cancer cell lines and may play a role in promoting cell migration by controlling the expression of APC and E-cadherin-catenin complexes (see Efstathiou et al., Proc. Natl. Acad. Sci. USA, Vol. 95, pp. 3122-3127 (1998)). As discussed previously, TFF1 is a fairly well-established predictive biomarker for estrogen therapy responsiveness and TFF1 mRNA levels are reportedly increased by estradiol but not by progesterone, dexamethasone or dihydrotestosterone (see Prud'homme et al., DNA, Vol. 4, pp. 11-21 (1985)). Furthermore, estradiol induction of TFF1 is reportedly inhibited by tamoxifen (see Prud'homme, supra.)

Another gene that co-clusters with ESR1, i.e., hepatocyte nuclear factor 3, alpha (HNF3A) activates TFF1 (see Beck et al., DNA Cell Biol., Vol. 18, pp. 157-164 (1999)). HNF3A was shown previously to co-cluster with ESR1 in expression profiles from 65 breast tumors by Perou et al., Nature, Vol. 406, pp. 747-752 (2000). Three additional genes listed in Table 1 also co-clustered with ESR1 in the report by Perou et al., supra: LIV-1; hepsin (HPN) a transmembrane protease which plays an essential role in cell growth and maintenance of cell morphology; and X-box binding protein 1 (XBP1) which binds to the HLA-DR-alpha promoter and may act as a transcription factor in B-cells (see Liou et al., Science, Vol. 247, pp. 1581-1584 (1990)).

AZGP1 is unique among the genes co-clustering with ESR1 in that it has not previously been associated with estrogen responsiveness but it has been considered as a biochemical marker of differentiation in breast cancer (see Diez-Itza et al., Eur. J. Cancer, Vol. 29A, pp. 1256-1260 (1993)). AZGP1 is a secreted protein that stimulates lipid degradation in adipocytes and may contribute to the extensive fat loss in patients with advanced cancer. It has high similarity to the extracellular domain of the alpha chain of class I MHC antigens.

Global analysis of gene expression at the mRNA level is a powerful tool for studying complex biological problems such as breast cancer. Here, clustering using standard correlation algorithms for expression array data was able to identify genes regulated with the ESR1. Eighteen genes were found, including 11 genes known to be ESR1-regulated or associated with breast cancer tumorigenesis. Interestingly, 4 of the genes present in the ESR1 branch described here, LIV1, HPN, XBP1 and HNF3A, were identified as members of a luminal epithelial ESR1 gene cluster described by Perou et al., Nature, Vol. 406, pp. 747-752 (2000)). XBP1 was also associated with ESR1 status in a third report of gene expression profiling of breast tumors by Bertucci et al., Hum. Mol. Genet., Vol. 9, pp. 2981-2991 (2000)). The co-clustering of HPN, HNF3A and XBP1 with ESR1 suggests that these genes, like LIV1, are regulated by estrogen and should be considered as possible markers for an intact ER-signaling pathway.

This is the first report of an association between ER and the following seven genes: SCNN1A, SERPINA3, ASAH, LCN1, TGFBR3, GRIA2 and CYP2B. The genes TGFBR3 and LCN1 are involved in cellular differentiation and proliferation and their de-regulation in a particular cell lineage that is also ESR1-positive in origin could result in tumorigenesis and co-clustering of ESR1 with these genes (see Bratt, Biochim. Biophys. Acta., Vol. 1482, pp. 318-326 (2000)).

Table 1 shows the genes that co-cluster with ESR1 in a hierarchical clustering of 1126 genes in 53 breast tumor biopsies, 1 normal breast and 5 cell line samples. The GenBank accession numbers shown for each gene are the accession numbers for the sequences from which the 25-mer probes used on the Affymetrix GeneChip are obtained for detection of that gene. Genes that have previously been shown to have expression that is positively correlated with ER are indicated by +.

TABLE 1
Genes that Co-Cluster with ESR1

| | | GenBank | |
|---|---|---|---|
| | Gene | Accession No. | Known Association with ESR1 |
| 1. | SCNN1A | X76180 | − |
| 2. | SERPINA3 | X68733 | − |
| 3. | ASAH | U70063 | − |
| 4. | LCN1 | L14927 | − |
| 5. | TGFBR3 | L07594 | − |
| 6. | GRIA2 | L20814 | − |
| 7. | CYP2B | M29874 | − |
| 8. | CEACAM5 | M29540 | + |
| 9. | MGB1 | U33147 | + |
| 10. | LIV1 | U41060 | + |
| 11. | PIP | HG1763 | + |
| 12. | MGP | X53331 | + |
| 13. | TFF3 | L08044 | + |
| 14. | TFF1 | X52003 | + |
| 15. | HNF3A | U39840 | + |
| 16. | HPN | X07732 | + |
| 17. | XBP1 | M31627 | + |
| 18. | AZGP1 | X59766 | − |
| 19. | ESR1 | X03635 | + |

Predictive Markers for Endocrine Responsivness in Pre-Treatment Biopsies

In another aspect of the invention 136 breast biopsies from 53 patients were obtained. RNA was extracted from 116 biopsies. Expression profiles were generated for 43 biopsies from 35 patients. Predictive markers of endocrine therapy responsiveness in breast tumors were identified. The breakdown of the profiled biopsies from the pre-letrozole (FEMARA™) treatments and the patient's clinical outcome was as follows: four patients with CR, nine patients with PR, four patients with NC and four patients with PD.

For the group treated with tamoxifen there were no patients in the CR category, 10 patients with PR, seven patients with NC and four patients with PD.

Patients with CR or PR were classified as “Responders” and those with NC or PD were classified as “Non-responders”. The expression of 8,000 genes was compared between these two groups in the pre-treatment biopsies from patients given Letrozole (FEMARA™). Numerical values (AvDiff) represent the expression level for that gene in a particular sample. For computational reasons the average of the AvDiff values was calculated for each gene on the array for all of the responders. These averages were then compared to each gene for each individual sample in the Non-responders group. Two genes were identified that had a three-fold or greater expression difference between the average of the Responders and each of the Non-responder samples, NOVA1 and IGHG3, both listed in Tables 2 and 6. Table 2 also includes V5 biopsy (post-treatment) data for reference only.

The two genes, IGHG3 and NOVA1, were found to be expressed at higher levels in the pre-treatment tumors from women who then ultimately responded positively to FEMARA™ treatment compared to biopsies from women who had NC or PD during FEMARA™ treatment. For the gene NOVA1, the difference in the median values between the two groups, including the V5 samples, is greater than would be expected by chance (P=0.012) using a Mann-Whitney Rank Sum Test. The data is not statistically significant for the gene IGHG3. These genes (IGHG3 and NOVA1) were not differentially expressed in biopsies from tamoxifen-treated patients and thus do not provide markers for favorable response to tamoxifen.

To uniquely identify the NOVA1 gene the following identifiers can be used: NOVA1 (Unigene ID Hs. 214) is located on chromosome 14q and is identified by the mRNA accession number of NM_{—}002515 and the protein accession number NP_{—}002506.

For the IGHG3 gene (Hs. 300697) this gene is also located on chromosome 14q and is identified by mRNA accession BC016381. There is no protein accession number.

There are several biological features of the genes, IGHG3 and NOVA1, that make these genes suitable as diagnostic markers and/or therapeutic targets. IGHG3 is associated with Heavy Chain Disease (HCD). HCD is a naturally occurring lymphoproliferative disease in which variant monoclonal Ig heavy (H) chain fragments are found in serum or urine. NOVA1 is a nuclear RNA binding protein with tightly regulated expression that is restricted to the neurons of the CNS in developing mice. Antibodies against this antigen are seen in paraneoplastic opsoclonus-ataxia (POA) patients. POA is an autoimmune disorder in which abnormal motor control of the eyes, trunk and limbs develops in women with breast or small lung cancer. Breast tumors in this disease aberrantly express the NOVA1 gene. This illicits an immune response that attacks the CNS which naturally expresses NOVA1. Serum reactivity with NOVA1 fusion protein is diagnostic for POA and suggests the presence of occult breast, gynecological or lung tumors.

TABLE 2
Genes with Variable Expression in Pre-Treatment (FEMARA ™) Breast
Biopsies from Patients That Responded Compared to Non-Responders

| | RESPONDERS (CR + PR) | | | | | | | | | | | | |
|---|---|---|---|---|---|---|---|---|---|---|---|---|---|
| | V0 | | | | | | | V5 | | | | | |
| PG | P380-2f | p382f | p141f | p610f | p615f | p611f | p580f | p387f | p592f | p143f | p111-2f | p582f | p598f |
| Sample | | | ▴ | | | | | | | ▴ | | | ▴ |
| No. | | | | | | | | | | | | | |
| IGHG3 | 19845 | 260.5 | 682.1 | 1551 | 2607 | 1051 | 18 | 128.8 | 631.4 | 2050 | 2869 | 2424 | 707.1 |
| | P | P | P | P | P | P | A | A | P | P | P | P | P |
| NOVA1 | 118.5 | 325.2 | 33.9 | 158.5 | 250.8 | 130.5 | 730 | 377.6 | 395.8 | 24.9 | 94.2 | 431.1 | 20.8 |
| | A | P | P | P | P | P | P | P | P | A | P | P | A |

| | NON-RESPONDERS (NC + PD) | | | | | | | |
|---|---|---|---|---|---|---|---|---|
| | V0 | | | | V5 | | | |
| PG | p568f | p136-2f | p609f | p613f | p391f | p589f | p566-2f | p5702f |
| Sample | ▴ | | | | | | | ▴ |
| No. | | | | | | | | |
| IGHG3 | 630.1 | 119.4 | 532.9 | 491.6 | 1451 | 1974 | 2833 | 5351 |
| | P | A | P | P | P | P | P | P |
| NOVA1 | 36.9 | 7.1 | 51.3 | 51.3 | 13.4 | 87.4 | 57.6 | 27.2 |
| | A | A | P | P | A | P | P | A |

PG Sample No. = a unique patient identifier.
V0 = biopsies taken at the first visit (pre-treatment).
V5 = The fifth visit (post-treatment).
▴= Found to be ER-based on gene expression profiling and ICH.
Numerical values (AvDiff) = the expression level for that gene in a particular sample.
Absolute call (AbsCall) = whether a gene is expressed in a sample or not is made by the Affymetrix software and is represented by A (absent; M (marginal); or P (present)

Predictive Markers From Post-Treatment Biopsies

In a further aspect of the invention, markers of responsiveness from post-treated patients were identified. For this purpose biopsies from letrozole (FEMARA™)-treated patients, the samples from V5, i.e., post-treatment biopsies, were placed into one of two categories, Responders or Non-Responders. Biopsies from patients that had CR or PR were considered to be Responders and those with NC or PD was classified as Non-Responders. For computational reasons the average of the AvgDiff values was calculated for each gene on the array for the V5 Responders. These averages were then compared to each gene for each individual sample in the Non-Responders group. Seven genes represented by 8 probe sets were identified as having a greater than three-fold difference in expression between the average of the Responders and each one of the samples in the Non-Responders group (Table 3). Table 3 also includes data from pre-treatment biopsies V0 for reference only. Two different probe sets for beta hemoglobin suggest that biopsies from patients that responded to FEMARA™ had a higher expression of this gene as compared to biopsies from Non-Responders. Interestingly, 2 genes identified, HPN and PIP, co-cluster with ESR1 in a 2-dimensional hierarchical clustering of ER-positive and ER-negative biopsies by gene expression. HPN (P=0.046) and lactotransferrin (P=<0.001) have a statistically significant difference in the median values between the Responders and Non-Responders using a Mann-Whitney Rank Sum Test. To perform the Mann-Whitney Rank Sum Test all biopsy data was used including V0 and V5 biopsies.

The list of markers includes HPN and PIP. These genes were also found to co-cluster with ESR1 in the hierarchical clustering analysis. Based on two separate analyses HPN and PIP should be considered as biomarkers of a functional ER transcriptosome that would be useful for predicting responsiveness to letrozole (FEMARA™).

HPN is a Type II, membrane-associated serine protease that has been shown to activate human factor VII and to initiate a pathway of blood coagulation on the cell surface leading to thrombin formation as described, e.g., in Kazama, J. Biol. Chem., Vol. 270, pp. 66-72 (1995). It is believed that a number of neoplastic cells activate the blood coagulation system, resulting in hypercoagulability and intravascular thrombosis through this and other pathways, and that hepsin plays a role in their cell growth, as described, e.g., in Torres-Rosada et al., Proc. Natl. Acad. Sci. USA, Vol. 90, pp. 7181-7185 (1993). The expression of the HPN gene is highly restricted; i.e., the gene is lowly-expressed in most body tissues with the exception of high levels in liver and moderate levels in the kidney as described, e.g., in Tsuji et al., J. Biol. Chem., Vol. 266, pp. 16948-16953 (1991).

HPN has been reported as highly-expressed in several cancer cell lines and, most recently, in ovarian cancer as described, e.g., in Tanimoto et al., Cancer Res., Vol. 57, pp. 2884-2887 (1997). In addition, although expression of HPN is high in the liver, knockout mice with disruptions in both copies of the HPN gene do not show liver abnormalities or dysfunction. Indeed, these mice do not show any discernable phenotype as described, e.g., in Wu et al., J. Clin. Invest., Vol. 101, pp. 321-6 (1998). Antibodies targeted against the extracellular domain of HPN have been shown to retard the growth of hepatoma cells that overexpress HPN as described, e.g., in Torres-Rosada et al., supra.

Two probes for beta hemoglobin were identified. This suggests that beta hemoglobin is more highly-expressed in Responders vs. Non-Responders in post-treatment (V5) tumors. It is possible that Letrozole (FEMARA™) targets well-vascularized breast tumors more successfully compared to poorly vascularized tumors and that beta hemoglobin expression levels correlate with the degree of vascularization in these biopsies. Lactotransferrin (LTF) was also included in the list of potential markers. LTF is an iron-binding protein expressed in milk that is also expressed in secondary granules of neutrophils. LTF is involved in iron transport storage and chelation, and host defense mechanisms. It was reported to be absent in ˜50% of breast tumors assayed (see Perou et al., Nature, Vol. 406, pp. 747-752 (200).

TABLE 3
Genes Found to Be Expressed At a Higher Level in Those Subjects Whose
Tumors Responded Positively to FEMARA ™ As Compared to Those
Subjects Who Did Not Respond Positively to FEMARA ™ Treatment

| | |
|---|---|
| 1 | Hepsin transmembrane protease, serine 1 |
| 2 | Hemoglobin beta |
| 3 | Hemoglobin beta |
| 4 | Glutamate receptor, ionotropic, AMPA2 |
| 5 | Tumor differentially expressed 1 |

TABLE 4
Genes Found to be Expressed At a Lower Level in Those Subjects Whose
Tumors Responded Positively to FEMARA ™ as Compared to Those
Subjects Who Did Not Respond Positively to FEMARA ™ Treatment

| | |
|---|---|
| 1 | Lactrotransferrin |
| 2 | Prolactin-induced protein (PIP)a |
| 3 | Sorbitol dehydrogenase |

Thus, the absolute levels of expression of these genes or their gene products can be measured in subjects who respond to Femara and in those who do not respond to Femara by any reliable means, including, but not limited to, the means disclosed herein, and the results compared to the expression levels of the same genes or gene products in an unknown subject to determine whether or not the unknown tumor will respond to endocrine therapy, including treatment with letrozole (FEMARA™).

TABLE 5
Genes with Variable Expression in Breast Biopsies from FEMARA ™
Responders Compared to Non-Responders

| | RESPONDERS (CR + PR) | | | | | | | | | | | | |
|---|---|---|---|---|---|---|---|---|---|---|---|---|---|
| | V0 | | | | | | | V5 | | | | | |
| PG | P380-2f | p382f | p141f | p610f | p615f | p611f | p580f | p111-2f | p143f | p387f | p582f | p592f | p598f |
| Sample | | | ▴ | | | | | | ▴ | | | | ▴ |
| No. | | | | | | | | | | | | | |
| HPN | 164 | 376.8 | −54.6 | 190.3 | 464.5 | 83.6 | 570.6 | −31.6 | 355.2 | 139 | 222 | 322.1 | −62.7 |
| | A | P | A | P | P | P | P | A | P | P | P | P | A |
| HBB | 85514 | 13307 | 6938 | 3738 | 686 | 3650 | 4009 | 37031 | 1900.4 | 7464 | 893 | 3907 | 241.7 |
| | P | P | P | P | P | P | P | P | P | P | P | P | P |
| M25079 | 2978 | 13979 | 5459 | 421 | 412 | 1737 | 924 | 28020.3 | 937.2 | 5607 | 406 | 506.4 | −3.8 |
| HBB | P | P | P | P | P | P | P | P | P | P | P | P | A |
| GRIA2 | −67.5 | 2307 | 5.1 | 76.7 | 2343 | 37.2 | 695 | 145 | 36.2 | 1334.6 | 221 | 31 | 2 |
| | A | P | A | P | P | P | P | P | A | P | P | P | A |
| LTF | 606 | 93.2 | −49.4 | −179.5 | 2.6 | 163.7 | 65.3 | −96.9 | 1896.8 | 959.3 | 192.4 | 154.2 | −38.5 |
| | A | A | A | A | A | P | A | A | P | P | P | P | A |
| PIP | 273.7 | 6817 | 20.6 | 0.9 | 1087 | 166.1 | 7703 | 3261.2 | 9095.4 | 8440.9 | 3473.9 | 7487.7 | 401.5 |
| | A | P | A | A | P | P | P | P | P | P | P | P | P |
| SORD | −15.3 | 539.1 | 95.8 | 206 | 2083 | 303.8 | 498.5 | 119.2 | 1413.9 | 865.1 | 865.9 | 1037 | 366 |
| | A | P | P | P | P | P | P | P | P | P | P | P | P |
| TDE1 | −107 | 273.2 | 150.2 | 209.8 | 291.7 | 196.9 | 161.9 | 130.5 | 187.1 | 444.4 | 268.3 | 58.4 | 209.7 |
| | A | P | P | P | P | P | P | P | P | P | P | A | P |

| | NON-RESPONDERS (NC + PD) | | | | | | | |
|---|---|---|---|---|---|---|---|---|
| | V0 | | | | V5 | | | |
| PG | p568f | p136-2f | p609f | p613f | p391f | p589f | p566-2f | p570-2f |
| Sample | ▴ | | | | | | | ▴ |
| No. | | | | | | | | |
| HPN | 37.7 | 162 | −52.5 | 79.3 | 40 | −23.6 | −53.1 | 20 |
| | A | P | A | P | A | A | A | A |
| HBB | 2627 | 16028 | 984 | 1590 | 692.6 | 1909.9 | 492.8 | 288 |
| | P | P | P | P | P | P | P | P |
| M25079 | 506 | 16030 | 161 | 247 | 285.7 | 438.2 | 53.4 | 39 |
| HBB | P | P | A | P | A | P | A | A |
| GRIA2 | 6.6 | 9.1 | 11.6 | 9.1 | 2.2 | 7.1 | 22.2 | 62 |
| | A | A | A | A | A | A | A | A |
| LTF | 7002 | 1318 | 525 | 698 | 2209.5 | 4953.1 | 5142.6 | 2592.2 |
| | P | P | P | P | P | P | P | P |
| PIP | 325.5 | 6922 | 3353 | 16.4 | 381.8 | 101.8 | 166.8 | 346 |
| | P | P | P | A | P | P | P | P |
| SORD | 113.2 | 494 | 1070 | 383 | 47.4 | 211.2 | 110.3 | 71.2 |
| | P | P | P | P | A | P | P | P |
| TDE1 | 104.1 | 35.8 | 467 | 38.6 | 51.3 | 57 | 20.4 | −30.6 |
| | P | P | P | P | P | P | A | A |

PG Sample No. = a unique patient identifier.
V0 = biopsies taken at the first visit (pre-treatment).
V5 = The fifth visit (post-treatment).
▴= Found to be ER-based on gene expression profiling and ICH.
Numerical values (AvDiff) = the expression level for that gene in a particular sample.
Absolute call (AbsCall) = whether a gene is expressed in a sample or not is made by the Affymetrix software and is represented by A (absent); M (marginal); or P (present).

TABLE 6
The Unigene Cluster Number For the Complete Genomic Sequence For All
the Genes Disclosed in This Application Except For IGHG3 and PIP For Which Only
Mrna Sequence is Available
The table also has the HUGO gene symbol and the protein accession number for the protein
expressed by the gene.

| | GenBank | | | |
|---|---|---|---|---|
| | Accession Number | Unigene | | Protein |
| | (used to design | Cluster | Gene | accession |
| Gene | Affymetrix Probes) | Number | Symbol | number |
| Sodium channel, nonvoltage-gated | X76180 | Hs.2794 | SCNN1A | prf:2015190A |
| 1 alpha | | | | |
| Serine or cysteine proteinase | X68733 | Hs.234726 | SERPINA | NA |
| inhibitor, member 3 | 3 | | | |
| N-acylsphingosine amidohydrolase | U70063 | Hs.75811 | ASAH | sp:Q13510 |
| (acid ceramidase) | | | | |
| Lipocalin 1 | L14927 | Hs.2099 | LCN1 | prf:1908211A |
| Transforming growth factor-beta | L07594 | Hs.79059 | TGFBR3 | sp:Q03167 |
| type III receptor | | | | |
| Glutamate receptor precursor 2 | L20814 | Hs.89582 | GRIA2 | pir:I58181 |
| Ctochrome P450-IIB, phenobarbital- | M29874 | Hs.1360 | CYP2B | pir:A32969 |
| inducible | | | | |
| Carcinoembryonic antigen mRNA | M29540 | Hs.220529 | CEACAM5 | pir:A36319 |
| Mammaglobin 1 | U33147 | Hs.46452 | MGB1 | sp:Q13296- |
| Estrogen regulated LIV-1 protein | U41060 | Hs.79136 | LIV-1 | pir:G02273 |
| Prolactin induced protein | HG1763 | Hs.99949 | PIP | pir:SQHUAC |
| Matrix Gla protein | X53331 | Hs.279009 | MGP | pir:GEHUM |
| Trefoil factor 3 | L08044 | Hs.82961 | TFF3 | sp:Q07654 |
| Trefoil factor 1 | X52003 | Hs.1406 | TFF1 | pir:A26667 |
| Hepatocyte nuclear factor-3 alpha | U39840 | Hs.299867 | HNF3A | pir:S70357 |
| Serine protease hepsin | X07732 | Hs.823 | HPN | pir:S00845 |
| X box binding protein-1 | M31627 | Hs.149923 | XBP1 | sp:P17861 |
| Zn-alpha2-glycoprotein | X59766 | Hs.71 | AZGP1 | pdb:1ZAG |
| Estrogen receptor alpha | X03635 | Hs.1657 | ESR1 | pir:S64737 |
| X-box binding protein 1 | M31627 | Hs.149923 | XBP1 | sp:P17861 |
| Neuro-oncological ventral antigen 1 | U04840 | Hs.214 | NOVA1 | pir:I38489 |
| Immunoglobulin heavy constant | M87789 | Hs.300697 | IGHG3 | NA |
| gamma 3 (G3m marker) | | | | |
| Hemoglobin beta | M25079 | Hs.155376 | HBB | prf:1701384A |
| Glutamate receptor ionotropic | L20814 | Hs.89582 | GRIA2 | pir:158181 |
| Lactotransferrin | X53961 | Hs.105938 | LTF | pir:TFHUL |
| Sorbitol dehydrogenase | L29008 | Hs.878 | SORD | sp:Q00796 |
| Tumor differentially expressed d 1 | U49188 | Hs.272168 | TDE1 | NA |

Pharmacoqenomics

Pharmacogenetics/genomics is the study of genetic/genomic factors involved in an individuals' response to a foreign compound or drug. Agents or modulators which have a stimulatory or inhibitory effect on expression of a marker of the invention can be administered to individuals to treat (prophylactically or therapeutically) breast cancer in the patient. In conjunction with such treatment, the pharmacogenomics of the individual must be considered. Differences in metabolism of therapeutics can lead to severe toxicity or therapeutic failure by altering the relation between dose and blood concentration of the pharmacologically active drug. Thus, understanding the pharmacogenomics of an individual permits the selection of effective agents (e.g., drugs) for prophylactic or therapeutic treatments. Such pharmacogenomics can further be used to determine appropriate dosages and therapeutic regimens. Accordingly, the level of expression of a marker of the invention in an individual can be determined to thereby select appropriate agent(s) for therapeutic or prophylactic treatment of the individual.

Pharmacogenomics deals with clinically significant variations in the efficacy or toxicity of drugs due to variations in drug disposition and action in individuals (see, e.g., Linder, Clin. Chem., Vol. 43, No. 2, pp. 254-266 (1997). In general, two types of pharmacogenetic conditions can be differentiated. Genetic conditions transmitted as a single factor altering the way drugs act on the body are referred to as “altered drug action”. Genetic conditions transmitted as single factors altering the way the body acts on drugs are referred to as “altered drug metabolism”. These pharmacogenetic conditions can occur either as rare defects or as common polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug.

These polymorphisms are expressed in two phenotypes in the population: the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme is the so-called ultra-rapid metabolizers who do not respond to standard doses. Recently, the molecular basis of ultra-rapid metabolism has been identified to be due to CYP2D6 gene amplification.

Thus, the level of expression, or the level of function, of a marker of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes, or drug targets to predict an individuals' drug responsiveness phenotype. This knowledge, when applied to dosing or drug selection, can avoid adverse reactions or therapeutic failure, and thus enhance therapeutic or prophylactic efficiency when treating a subject with a modulator of expression of a marker of the invention.

Proteomics

Proteins that are secreted by both normal and transformed cells in culture can be analyzed to identify those proteins that are likely to be secreted by cancerous cells into body fluids and may be of value in the methods of this invention. Supernatants can be isolated and MWT-CO filters can be used to simplify the mixture of proteins. The proteins can then be digested with trypsin. The tryptic peptides may then be loaded onto a microcapillary HPLC column where they are separated, and eluted directly into an ion trap mass spectrometer, through a custom-made electrospray ionization source. Throughout the gradient, sequence data can be acquired through fragmentation of the four most intense ions (peptides) that elute off the column, while dynamically excluding those that have already been fragmented. In this way, the sequence data from multiple scans can be obtained, corresponding to approximately 50-200 different proteins in the sample. These data are searched against databases using correlation analysis tools, such as MS-Tag, to identify the proteins in the supernatants.

Measurement Methods

The experimental methods of this invention depend on measurements of cellular constituents. The cellular constituents measured can be from any aspect of the biological state of a cell. They can be from the transcriptional state, in which RNA abundances are measured, the translation state, in which protein abundances are measured, the activity state, in which protein activities are measured. The cellular characteristics can also be from mixed aspects, for example, in which the activities of one or more proteins are measured along with the RNA abundances (gene expressions) of other cellular constituents. This section describes exemplary methods for measuring the cellular constituents in drug or pathway responses. This invention is adaptable to other methods of such measurement.

Preferably, in this invention the transcriptional state of the other cellular constituents is measured. The transcriptional state can be measured by techniques of hybridization to arrays of nucleic acid or nucleic acid mimic probes, described in the next subsection, or by other gene expression technologies, described in the subsequent subsection. However measured, the result is data including values representing mRNA abundance and/or ratios, which usually reflect DNA expression ratios (in the absence of differences in RNA degradation rates).

In various alternative embodiments of the present invention, aspects of the biological state other than the transcriptional state, such as the translational state, the activity state, or mixed aspects can be measured.

In one aspect of the invention the presence, progression or prognosis of breast cancer in a subject can be monitored by measuring a level of expression of mRNA or encoded protein corresponding to at least one of the genes identified in Tables 1, 2, 3 or 4 in a sample of bodily fluid or breast tissue obtained in the subject over time, i.e., at various stages of the breast disorder. The level of expression of the mRNA or encoded protein corresponding to the gene(s) identified as relevant to overall prognosis can provide valuable information concerning the treatment or progression of the breast cancer. The level of expression of mRNA and protein corresponding to the gene(s) can be detected by standard methods as described below.

In a particularly useful embodiment, the level of mRNA expression of a plurality of the disclosed genes can be measured simultaneously in a subject at various stages of the breast disorder to generate a transcriptional or expression profile of the breast disorder over time. For example, mRNA transcripts corresponding to a plurality of these genes can be obtained from breast cells of a subject at different times, and hybridized to a chip containing oligonucleotide probes which are complementary to the transcripts of the desired genes, to compare expression of a large number of genes at various stages of the breast cancer.

In another aspect, a cell-based assay based on the disclosed genes can be used to identify agents for use in the treatment of breast cancer. This method comprises: a) contacting a sample of bodily fluid or breast tissue obtained from a subject suspected of having a breast disorder with a candidate agent; b) detecting a level of expression of at least one gene identified in Tables 1, 2, 3 or 4; and c) comparing the level of expression of the gene in the sample in the absence of the candidate agent, wherein a change in the level of expression in the sample in the presence of the agent relative to the level of expression in the absence of the agent is indicative of an agent useful in the treatment of a breast cancer. The level of expression of the gene is detected by measuring the level of mRNA corresponding to, or protein encoded, by the gene as described below.

As used herein the term “similar”, when applied to a comparison of two or more values, means that the values are within 10% of each other.

As used herein, the term “candidate agent” refers to any molecule that is capable of altering or decreasing the level of mRNA corresponding to, or protein encoded, by at least one of the disclosed genes. The candidate agent can be natural or synthetic molecules such as proteins or fragments thereof, antibodies, small molecule inhibitors, nucleic acid molecules, e.g., antisense nucleotides, ribozymes, double-stranded RNAs, organic and inorganic compounds and the like.

Cell-free assays can also be used to identify compounds which are capable of interacting with a protein encoded by one of the disclosed genes or protein binding partner, to alter the activity of the protein or its binding partner. Cell-free assays can also be used to identify compounds, which modulate the interaction between the encoded protein and its binding partner such as a target peptide.

In one embodiment, cell-free assays for identifying such compounds comprise a reaction mixture containing a protein encoded by one of the disclosed genes and a test compound or a library of test compounds in the presence or absence of the binding partner, e.g., a biologically inactive target peptide or a small molecule. Accordingly, one example of a cell-free method for identifying agents useful in the treatment of breast cancer is provided which comprises contacting a protein or functional fragment thereof or the protein binding partner with a test compound or library of test compounds and detecting the formation of complexes. For detection purposes, the protein can be labeled with a specific marker and the test compound or library of test compounds labeled with a different marker. Interaction of a test compound with the protein or fragment thereof or the protein binding partner can then be detected by measuring the level of the two labels after incubation and washing steps. The presence of the two labels is indicative of an interaction.

Interaction between molecules can also be assessed by using real-time BIA (Biomolecular Interaction Analysis, Pharmacia Biosensor (AB) which detects surface plasmon resonance, an optical phenomenon. Detection depends on changes in the mass concentration of mass macromolecules at the biospecific interface and does not require labeling of the molecules. In one useful embodiment, a library of test compounds can be immobilized on a sensor surface, e.g., a wall of a micro-flow cell. A solution containing the protein, functional fragment thereof, or the protein binding partner is then continuously circulated over the sensor surface. An alteration in the resonance angle, as indicated on a signal recording, indicates the occurrence of an interaction. This technique is described in more detail in BIAtechnology Handbook by Pharmacia.

Another embodiment of a cell-free assay comprises: a) combining a protein encoded by the at least one gene, the protein binding partner and a test compound to form a reaction mixture; and b) detecting interaction of the protein and the protein binding partner in the presence and absence of the test compounds. A considerable change (potentiation or inhibition) in the interaction of the protein and binding partner in the presence of the test compound compared to the interaction in the absence of the test compound indicates a potential agonist (mimetic or potentiator) or antagonist (inhibitor) of the proteins' activity for the test compound. The components of the assay can be combined simultaneously or the protein can be contacted with the test compound for a period of time, followed by the addition of the binding partner to the reaction mixture. The efficacy of the compound can be assessed by using various concentrations of the compound to generate dose response curves. A control assay can also be performed by quantitating the formation of the complex between the protein and its binding partner in the absence of the test compound.

Formation of a complex between the protein and its binding partner can be detected by using detectably labeled proteins such as radiolabeled, fluorescently-labeled or enzymatically-labeled protein or its binding partner, by immunoassay or by chromatographic detection.

In preferred embodiments, the protein or its binding partner can be immobilized to facilitate separation of complexes from uncomplexed forms of the protein and its binding partner and automation of the assay. Complexation of the protein to its binding partner can be achieved in any type of vessel, e.g., microtitre plates, micro-centrifuge tubes and test tubes. In particularly preferred embodiment, the protein can be fused to another protein, e.g., glutathione-S-transferase to form a fusion protein which can be absorbed onto a matrix, e.g., glutathione sepharose beads (Sigma Chemical, St. Louis, Mo.) which are then combined with the labeled protein partner, e.g., labeled with ³⁵S, and test compound and incubated under conditions sufficient to formation of complexes. Subsequently, the beads are washed to remove unbound label and the matrix is immobilized and the radiolabel is determined.

Another method for immobilizing proteins on matrices involves utilizing biotin and streptavidin. For example, the protein can be biotinylated using biotin NHS(N-hydroxy-succinimide) using well-known techniques and immobilized in the well of steptavidin-coated plates.

Cell-free assays can also be used to identify agents which are capable of interacting with a protein encoded by the at least one gene and modulate the activity of the protein encoded by the gene. In one embodiment, the protein is incubated with a test compound and the catalytic activity of the protein is determined. In another embodiment, the binding affinity of the protein to a target molecule can be determined by methods known in the art.

The present invention also provides for both prophylactic and therapeutic methods of treating a subject having, or at risk of having, a breast disorder. Administration of a prophylactic agent can occur prior to the manifestation of symptoms characteristic of the breast disorder, such that development of the breast disorder is prevented or delayed in its progression. With respect to treatment of the breast disorder, it is not required that the breast cell, e.g., cancer cell, be killed or induced to undergo cell death. Instead, all that is required to achieve treatment of the breast disorder is that the tumor growth be slowed down to some degree or that some of the abnormal cells revert back to normal. Examples of suitable therapeutic agents include, but are not limited to, antisense nucleotides, ribozymes, double-stranded RNAs and antagonists as described in detail below.

As used herein the term “antisense” refers to nucleotide sequences that are complementary to a portion of an RNA expression product of at least one of the disclosed genes. “Complementary” nucleotide sequences refer to nucleotide sequences that are capable of base-pairing according to the standard Watson-Crick complementary rules. That is, purines will base-pair with pyrimidine to form combinations of guanine:cytosine and adenine:thymine in the case of DNA, or adenine:uracil in the case of RNA. Other less common bases, e.g., inosine, 5-methylcytosine, 6-methyladenine, hypoxanthine and others may be included in the hybridizing sequences and will not interfere with pairing.

In all embodiments, measurements of the cellular constituents should be made in a manner that is relatively independent of when the measurements are made.

Transcriptional State Measurement

Preferably, measurement of the transcriptional state is made by hybridization of nucleic acids to oligonucleotide arrays, which are described in this subsection. Certain other methods of transcriptional state measurement are described later in this subsection.

Transcript Arrays Generally

In a preferred embodiment the present invention makes use of “oligonucleotide arrays” (also called herein “microarrays”). Microarrays can be employed for analyzing the transcriptional state in a cell, and especially for measuring the transcriptional states of cancer cells.

In one embodiment, transcript arrays are produced by hybridizing detectably labeled polynucleotides representing the mRNA transcripts present in a cell (e.g., fluorescently-labeled cDNA synthesized from total cell mRNA or labeled cRNA) to a microarray. A microarray is a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics. The arrays are reproducible, allowing multiple copies of a given array to be produced and easily compared with each other. Preferably the microarrays are small, usually smaller than 5 cm², and they are made from materials that are stable under binding (e.g., nucleic acid hybridization) conditions. A given binding site or unique set of binding sites in the microarray will specifically bind the product of a single gene in the cell. Although there may be more than one physical binding site (hereinafter “site”) per specific mRNA, for the sake of clarity the discussion below will assume that there is a single site. In a specific embodiment, positionally addressable arrays containing affixed nucleic acids of known sequence at each location are used.

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA or cRNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

Preparation of Microarrays

Microarrays are known in the art and consist of a surface to which probes that correspond in sequence to gene products (e.g., cDNAs, mRNAs, cRNAs, polypeptides and fragments thereof, can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA or cRNA can specifically hybridize. The nucleic acid or analogue of the binding site can be, e.g., a synthetic oligomer, a full-length cDNA, a less-than full-length cDNA, or a gene fragment.

Although in a preferred embodiment the microarray contains binding sites for products of all or almost all genes in the target organism's genome, such comprehensiveness is not necessarily required. The microarray may have binding sites for only a fraction of the genes in the target organism. However, in general, the microarray will have binding sites corresponding to at least about 50% of the genes in the genome, often at least about 75%, more often at least about 85%, even more often more than about 90%, and most often at least about 99%. Preferably, the microarray has binding sites for genes relevant to testing and confirming a biological network model of interest. A “gene” is identified as an open reading frame (ORF) of preferably at least 50, 75 or 99 amino acids from which a messenger RNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence. For example, the Saccharomyces cerevisiae genome has been completely sequenced and is reported to have approximately 6275 ORFs longer than 99 amino acids. Analysis of these ORFs indicates that there are 5885 ORFs that are likely to specify protein products (see, e.g., Goffeau et al., “Life with 6000 genes”, Science, Vol. 274, pp. 546-567 (1996)), which is incorporated by reference in its entirety for all purposes). In contrast, the human genome is estimated to contain approximately 25,000-35,000 genes.

Preparing Nucleic Acids for Microarrays

As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., polymerase chain reaction (PCR) amplification of gene segments from genomic DNA, cDNA (e.g., by RT-PCR), or cloned sequences or the sequences may be synthesized de novo on the surface of the chip, for example by use of photolithography techniques, e.g., Affymetrix uses such a different technology to synthesize their oligos directly on the chip). PCR primers are chosen, based on the known sequence of the genes or cDNA, that result in amplification of unique fragments (i.e., fragments that do not share more than 10 bases of contiguous identical sequence with any other fragment on the microarray). Computer programs are useful in the design of primers with the required specificity and optimal amplification properties (see, e.g., Oligo pl version 5.0 (National Biosciences)). In the case of binding sites corresponding to very long genes, it will sometimes be desirable to amplify segments near the 3′ end of the gene so that when oligo-dT primed cDNA probes are hybridized to the microarray; less-than-full length probes will bind efficiently. Typically each gene fragment on the microarray will be between about 20 bp and about 2000 bp, more typically between about 100 bp and about 1000 bp, and usually between about 300 bp and about 800 bp in length. PCR methods are well known and are described, for example, in Innis et al. Eds., “PCR Protocols: A Guide to Methods and Applications”, Academic Press Inc., San Diego, Calif. (1990), which is incorporated by reference in its entirety for all purposes. It will be apparent that computer controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries (Froehler et al., Nucleic Acid Res., Vol. 14, pp. 5399-5407 (1986); McBride et al., Tetrahedron Lett., Vol. 24, pp. 245-248 (1983)). Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, e.g., Egholm et al., “PNA Hybridizes to Complementary Oligonucleotides Obeying the Watson-Crick Hydrogen-Bonding Rules”, Nature, Vol. 365, pp. 566-568 (1993); see also U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al., “Differential Gene Expression in the Murine Thymus Assayed by Quantitative Hybridization of Arrayed cDNA Clones”, Genomics, Vol. 29, pp. 207-209 (1995)). In yet another embodiment, the polynucleotide of the binding sites is RNA.

Attaching Nucleic Acids to the Solid Surface

The nucleic acid or analogue are attached to a solid support, which may be made from glass, plastic (e.g., polypropylene, nylon), polyacrylamide, nitrocellulose or other materials. A preferred method for attaching the nucleic acids to a surface is by printing on glass plates, as is described generally by Schena et al., “Quantitative Monitoring of Gene Expression Patterns With a Complementary DNA Microarray, Science, Vol. 270, pp. 467-470 (1995)). This method is especially useful for preparing microarrays of cDNA. See, also, DeRisi et al., “Use of a cDNA Microarray to Analyze Gene Expression Patterns in Human Cancer”, Nature Genetics, Vol. 14, pp. 457-460 (1996); Shalon et al., “A DNA Microarray System for Analyzing Complex DNA Samples Using Two-Color Fluorescent Probe Hybridization, Genome Res., Vol. 6, pp. 639-645 (1996); and Schena et al., “Parallel Human Genome Analysis; Microarray-Based Expression of 1000 Genes”, Proc. Natl. Acad. Sci. USA, Vol. 93, pp. 10539-11286 (1995)). Each of the aforementioned articles is incorporated by reference in its entirety for all purposes.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see Fodor et al., “Light-Directed Spatially Addressable Parallel Chemical Synthesis”, Science, Vol. 251, pp. 767-773 (1991); Pease et al., “Light-Directed Oligonucleotide Arrays for Rapid DNA Sequence Analysis”, Proc. Natl. Acad. Sci. USA, Vol. 91, pp. 5022-5026 (1994); Lockhart et al., “Expression Monitoring by Hybridization to High-Density Oligonucleotide Arrays”, Nature Biotech., Vol. 14, p. 1675 (1996); U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al., “High-Density Oligonucleotide Arrays”, Biosensors & Bioelectronics, Vol. 11, pp. 687-690 (1996)). When these methods are used, oligonucleotides (e.g., 25 mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (see Maskos and Southern, Nuc. Acids Res., Vol. 20, pp. 1679-1684 (1992)), may also be used. In principal, any type of array, for example, dot blots on a nylon hybridization membrane (see Sambrook et al., “Molecular Cloning—A Laboratory Manual (2nd Ed.)”, Vols. 1-3, Cold Spring Harbor Laboratory, Cold Spring Harbor, N.Y. (1989), which is incorporated in its entirety for all purposes), could be used, although, as will be recognized by those of skill in the art, very small arrays will be preferred because hybridization volumes will be smaller.

Generating Labeled Probes

Methods for preparing total and poly(A)⁺ RNA are well-known and are described generally in Sambrook et al., supra. In one embodiment, RNA is extracted from cells of the various types of interest in this invention using guanidinium thiocyanate lysis followed by CsCI centrifugation (Chirgwin et al., Biochemistry, Vol. 18, pp. 5294-5299 (1979)). Poly(A)⁺ RNA is selected by selection with oligo-dT cellulose (see Sambrook et al., supra). Cells of interest include wild-type cells, drug-exposed wild-type cells, cells with modified/perturbed cellular constituent(s), and drug-exposed cells with modified/perturbed cellular constituent(s).

Labeled cDNA is prepared from mRNA or alternatively directly from RNA by oligo dT-primed or random-primed reverse transcription, both of which are well known in the art (see, e.g., Klug and Berger, Methods Enzymol., Vol. 152, pp. 316-325 (1987)). Reverse transcription may be carried out in the presence of a dNTP conjugated to a detectable label, most preferably a fluorescently-labeled dNTP. Alternatively, isolated mRNA can be converted to labeled antisense RNA synthesized by in vitro transcription of double-stranded cDNA in the presence of labeled dNTPs (see Lockhart et al., “Expression Monitoring by Hybridization to High-Density Oligonucleotide Arrays”, Nature Biotech., Vol. 14, p. 1675 (1996)), which is incorporated by reference in its entirety for all purposes. In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, “Nonisotopic DNA Probe Techniques”, Academic Press, San Diego, Calif. (1992)). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., “High Density cDNA Filter Analysis: A Novel Approach for Large-Scale, Quantitative Analysis of Gene Expression”, Gene, Vol. 156, p. 207 (1995); Pietu et al., “Novel Gene Transcripts Preferentially Expressed in Human Muscles Revealed by Quantitative Hybridization of a High Density cDNA Array”, Genome Res., Vol. 6, p. 492 (1996)). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., ™II, LTI Inc.) at 42° C. for 60 minutes.

Hybridization to Microarrays

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra, and Chee et al., supra). Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-Interscience, NY (1987), which is incorporated in its entirety for all purposes. When the cDNA microarrays of Schena et al. are used, typical hybridization conditions are hybridization in 5× SSC plus 0.2% SDS at 65° C. for 4 hours followed by washes at 25° C. in low stringency wash buffer (1× SSC plus 0.2% SDS) followed by 10 minutes at 25° C. in high stringency wash buffer (0.1× SSC plus 0.2% SDS) (see Shena et al., Proc. Natl. Acad. Sci. USA, Vol. 93, p. 10614 (1996)). Useful hybridization conditions are also provided in, e.g., Tijessen, “Hybridization With Nucleic Acid Probes”, Elsevier Science Publishers B. V. (1993) and Kricka, “Nonisotopic DNA Probe Techniques”, Academic Press, San Diego, Calif. (1992).

Signal Detection and Data Analysis

When fluorescently-labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows specimen illumination at wavelengths specific to the fluorophores used and emissions from the fluorophore can be analyzed. In a preferred embodiment, the arrays are scanned with a laser fluorescent scanner with a computer controlled X-Y stage and a microscope objective. Sequential excitation of the fluorophore is achieved with a multi-line, mixed gas laser and the emitted light is split by wavelength and detected with a photomultiplier tube. Fluorescence laser scanning devices are described in Schena et al., Genome Res., Vol. 6, pp. 639-645 (1996) and in other references cited herein. Alternatively, the fiber-optic bundle described by Ferguson et al., Nature Biotech., Vol. 14, pp. 1681-1684 (1996), may be used to monitor mRNA abundance levels at a large number of sites simultaneously.

Signals are recorded and, in a preferred embodiment, analyzed by computer, e.g., using a 12-bit analog to digital board. In one embodiment the scanned image is de-speckled using a graphics program (e.g., Hijaak Graphics Suite) and then analyzed using an image gridding program that creates a spreadsheet of the average hybridization at each wavelength at each site.

The Agilent Technologies GENEARRAY™ scanner is a bench-top, 488 nM argon-ion laser-based analysis instrument. The laser can be focused to a spot size of less than 4 microns. This precision allows for the scanning of probe arrays with probe cells as small as 20 microns. The laser beam focuses onto the probe array, exciting the fluorescent-labeled nucleotides. It then and then scans using the selected filter for the dye used in the assay. Scanning in the orthogonal coordinate is achieved by moving the probe array. The laser radiation is absorbed by the dye molecules incorporated into the hybridized sample and causes them to emit fluorescence radiation. This fluorescent light is collimated by a lens and passes through a filter for wavelength selection. The light is then focused by a second lens onto an aperture for depth discrimination and then detected by a highly sensitive photo multiplier tube (PMT). The output current of the PMT is converted into a voltage read by an analog to digital converter (ADC) and the processed data is passed back to the computer as the fluorescent intensity level of the sample point, or picture element (pixel) currently being scanned. The computer displays the data as an image, as the scan progresses. In addition, the fluorescent intensity level of all samples, representing the expression profile of the sample, is recorded in computer readable format.

If necessary, an experimentally determined correction for “cross talk” (or overlap) between the channels for the two fluors may be made. For any particular hybridization site on the transcript array, a ratio of the emission of the two fluorophores may be calculated. The ratio is independent of the absolute expression level of the cognate gene, but may be useful for genes whose expression is significantly modulated by drug administration, gene deletion, or any other tested event.

Preferably, in addition to identifying a perturbation as positive or negative, it is advantageous to determine the magnitude of the perturbation. This can be carried out by methods that will be readily apparent to those of skill in the art.

As used herein, the term “similar”, when used to compare two or more values, means that the two values are within 20%, or more preferably within 10% of each other in numerical value when using the same units.

Other Methods of Transcriptional State Measurement

The transcriptional state of a cell may be measured by other gene expression technologies known in the art. Several such technologies produce pools of restriction fragments of limited complexity for electrophoretic analysis, such as methods combining double restriction enzyme digestion with phasing primers (see, e.g., European Patent 0 534858 A1, filed Sep. 24,1992, by Zabeau et al.), or methods selecting restriction fragments with sites closest to a defined mRNA end (see, e.g., Prashar et al., Proc. Natl. Acad. Sci. USA, Vol. 93, pp. 659-663 (1996)). Other methods statistically sample cDNA pools, such as by sequencing sufficient bases (e.g., 20-50 bases) in each of multiple cDNAs to identify each cDNA, or by sequencing short tags (e.g., 9-10 bases) which are generated at known positions relative to a defined mRNA end (see, e.g., Velculescu, Science, Vol. 270, pp. 484-487 (1995)) pathway pattern.

Measurement of Other Aspects

In various embodiments of the present invention, aspects of the biological state other than the transcriptional state, such as the translational state, the activity state or mixed aspects can be measured in order to obtain drug and pathway responses. Details of these embodiments are described in this section.

Translational State Measurements

Expression of the protein encoded by the gene(s) can be detected by a probe which is detectably labeled, or which can be subsequently labeled. Generally, the probe is an antibody that recognizes the expressed protein.

As used herein, the term “antibody” includes, but is not limited to, polyclonal antibodies, monoclonal antibodies, humanized or chimeric antibodies, and biologically functional antibody fragments sufficient for binding of the antibody fragment to the protein.

For the production of antibodies to a protein encoded by one of the disclosed genes, various host animals may be immunized by injection with the polypeptide, or a portion thereof. Such host animals may include, but are not limited to, rabbits, mice and rats, to name but a few. Various adjuvants may be used to increase the immunological response, depending on the host species, including, but not limited to, Freund's (complete and incomplete), mineral gels such as aluminum hydroxide, surface active substances, such as lysolecithin, pluronic polyols, polyanions, peptides, oil emulsions, keyhole limpet hemocyanin, dinitrophenol and potentially useful human adjuvants such as BCG (bacille Camette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler and Milstein, Nature, Vol. 256, pp. 495-497 (1975); and U.S. Pat. No. 4,376,110. The human B-cell hybridoma technique of Kosbor et al., Immunology Today, Vol. 4, No. 72 (1983); Cole et al., Proc. Natl. Acad. Sci. USA, Vol. 80, pp. :2026-2030 (1983); and the EBV-hybridoma technique, Cole et al., Monoclonal Antibodies and Cancer Therapy, Alan R. Liss, Inc., pp. 77-96 (1985). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies”, Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985), by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived form a murine mAb and a human immunoglobulin constant region.

Alternatively, techniques described for the production of single chain antibodies, U.S. Pat. No. 4,946,778; Bird, Science, Vol. 242, pp. 423-426 (1988); Huston et al., Proc. Natl. Acad. Sci. USA, Vol. 85, pp. 5879-5883 (1988); and Ward et al., Nature, Vol. 334, pp. 544-546 (1989), can be adapted to produce differentially expressed gene-single chain antibodies. Single chain antibodies are formed by linking the heavy and light chain fragments of the Fv region via an amino acid bridge, resulting in a single chain polypeptide.

More preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments, which recognize specific epitopes, may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed, Huse et al., Science, Vol. 246, pp. 1275-1281 (1989), to allow rapid and easy identification of monoclonal Fab fragments with the desired specificity.

The extent to which the known proteins are expressed in the sample is then determined by immunoassay methods that utilize the antibodies described above. Such immunoassay methods include, but are not limited to, dot blotting, western blotting, competitive and non-competitive protein binding assays, enzyme-linked immunosorbant assays (ELISA), immunohistochemistry, fluorescence activated cell sorting (FACS) and others commonly used and widely described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested brought into contact with the bound molecule after a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody must be an antibody that is specific for the protein expressed by the gene of interest.

The most commonly used reporter molecules in this type of assay are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of protein which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well-established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Measurement of the translational state may also be performed according to several additional methods. For example, whole genome monitoring of protein (i.e., the “proteome”, Goffeau et al., supra) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. Methods for making monoclonal antibodies are well known (see, e.g., Harlow and Lane, “Antibodies: A Laboratory Manual”, Cold Spring Harbor, N.Y. (1988), which is incorporated in its entirety for all purposes). In a one preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension (see, e.g., Hames et al., “Gel Electrophoresis of Proteins: A Practical Approach”, IRL Press, NY (1990); Shevchenko et al., Proc. Nat'l Acad. Sci. USA, Vol. 93, pp. 1440-1445 (1996); Sagliocco et al., Yeast, Vol. 12, pp. 1519-1533 (1996); Lander, Science, Vol. 274, pp. 536-539 (1996). The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

Embodiments Based on Other Aspects of the Biological State

Although monitoring cellular constituents other than mRNA abundances currently presents certain technical difficulties not encountered in monitoring mRNAs, it will be apparent to those of skill in the art that the use of methods of this invention that the activities of proteins relevant to the characterization of cell function can be measured, embodiments of this invention can be based on such measurements. Activity measurements can be performed by any functional, biochemical, or physical means appropriate to the particular activity being characterized. Where the activity involves a chemical transformation, the cellular protein can be contacted with the natural substrates, and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances and changes in certain protein activities.

Computer Implementations

In a preferred embodiment, the computation steps of the previous methods are implemented on a computer system or on one or more networked computer systems in order to provide a powerful and convenient facility for forming and testing models of biological systems. The computer system may be a single hardware platform comprising internal components and being linked to external components. The internal components of this computer system include processor element interconnected with a main memory. For example computer system can be an Intel Pentium based processor of 200 Mhz or greater clock rate and with 32 MB or more of main memory.

The external components include mass data storage. This mass storage can be one or more hard disks (which are typically packaged together with the processor and memory). Typically, such hard disks provide for at least 1 GB of storage. Other external components include user interface device, which can be a monitor and keyboards, together with pointing device, which can be a “mouse”, or other graphic input devices. Typically, the computer system is also linked to other local computer systems, remote computer systems or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on mass storage. Alternatively, the software components may be stored on removable media such as floppy disks or CD-ROM (not illustrated). The software component represents the operating system, which is responsible for managing the computer system and its network interconnections. This operating system can be, e.g., of the Microsoft Windows family, such as Windows 95, Windows 98 or Windows NT, or a Unix operating system, such as Sun Solaris. Software includes common languages and functions conveniently present on this system to assist programs implementing the methods specific to this invention. Languages that can be used to program the analytic methods of this invention include C, C++, or, less preferably, JAVA. Most preferably, the methods of this invention are programmed in mathematical software packages, which allow symbolic entry of equations and high-level specification of processing, including algorithms to be used, and thereby freeing a user of the need to procedurally program individual equations or algorithms. Such packages include, e.g., MATLAB™ from Mathworks (Natick, Mass.), MATHEMATICA™ from Wolfram Research (Champaign, Ill.), and MATHCAD™ from Mathsoft (Cambridge, Mass.).

In preferred embodiments, the analytic software component actually comprises separate software components that interact with each other. Analytic software represents a database containing all data necessary for the operation of the system. Such data will generally include, but is not necessarily limited to, results of prior experiments, genome data, experimental procedures and cost, and other information, which will be apparent to those skilled in the art. Analytic software includes a data reduction and computation component comprising one or more programs which execute the analytic methods of the invention. Analytic software also includes a user interface (UI) which provides a user of the computer system with control and input of test network models, and, optionally, experimental data. The user interface may comprise a drag-and-drop interface for specifying hypotheses to the system. The user interface may also comprise means for loading experimental data from the mass storage component (e.g., the hard drive), from removable media (e.g., floppy disks or CD-ROM), or from a different computer system communicating with the instant system over a network (e.g., a local area network, or a wide area communication network, such as the internet).

This invention also provides a process for preparing a database comprising at least one of the markers set forth in this invention, e.g., mRNAs or protein products. For example, the polynucleotide or amino acid sequences are stored in a digital storage medium such that a data processing system for standardized representation of the genes that identify a breast cancer cell is compiled. The data processing system is useful to analyze gene expression between two cells by first selecting a cell suspected of being of a neoplastic phenotype or genotype and then isolating polynucleotides from the cell. The isolated polynucleotides are sequenced. The sequences from the sample are compared with the sequence(s) present in the database using homology search techniques. Greater than 90%, more preferably, greater than 95%, and more preferably, greater than, or equal to, 97%, sequence identity between the test sequence and the polynucleotides of the present invention, is a positive indication that the polynucleotide has been isolated from a breast cancer cell as defined above.

Alternative computer systems and methods for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

Methods of Modifying the Abundance or Activity of mRNA

In various embodiments of this invention altering or modifying the abundance or activity of expressed mRNA produces clinically beneficial effects. Methods of modifying RNA abundance and activities currently fall within four classes; ribozymes, antisense species, double-stranded RNA and RNA aptamers (Good et al., Gene Therapy, Vol. 4, pp. 45-54 (1997)). Controllable application or exposure of a cell to these entities permits controllable perturbation of RNA abundance including mRNA abundance and activity, including its translation into active or detectable gene expression products, i.e., proteins.

Ribozymes

Ribozymes are RNA molecules that specifically cleave other single-stranded RNA in a manner similar to DNA restriction endonucleases. Ribozymes are capable of catalyzing RNA cleavage reactions (Cech, Science, Vol. 236, pp. 1532-1539 (1987); PCT International Publication WO 90/11364, published Oct. 4, 1990; Sarver et al., Science, Vol. 247, pp. 1222-1225 (1990)). By modifying the nucleotide sequences encoding the RNAs, ribozymes can be synthesized to recognize specific nucleotide sequences in a molecule and cleave it as described, e.g., in Cech, Amer. Med. Assn., Vol. 260, pp. 3030 (1988). Accordingly, only mRNAs with specific sequences are cleaved and inactivated.

Two basic types of ribozymes include the “hammerhead”-type as described, for example, in Rossie et al., Pharmac. Ther., Vol. 50, pp. 245-254 (1991); and the “hairpin” ribozyme as described, e.g., in Hampel et al., Nucl. Acids Res., Vol. 18, pp. 299-304 (1999) and U.S. Pat. No. 5,254,678. Hairpin and hammerhead RNA ribozymes can be designed to specifically cleave a particular target mRNA. Rules have been established for the design of short RNA molecules with ribozyme activity, which are capable of cleaving other RNA molecules in a highly sequence specific way and can be targeted to virtually all kinds of RNA (Haseloff et al., Nature, Vol. 334, pp. 585-591 (1988); Koizumi et al., FEBS Left., Vol. 228, pp. 228-230 (1988); Koizumi et al., FEBS Left., Vol. 239, pp. 285-288 (1988)).

Ribozyme methods involve exposing a cell to, inducing expression in a cell, etc. of such small RNA ribozyme molecules (Grassi and Marini, Annals of Medicine, Vol. 28, pp. 499-510 (1996); Gibson, Cancer and Metastasis Reviews, Vol. 15, pp. 287-299 (1996)). Intracellular expression of hammerhead and hairpin ribozymes targeted to mRNA corresponding to at least one of the disclosed genes can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (see Cotten et al., “Ribozyme Mediated Destruction of RNA In Vivo”, The EMBO J., Vol. 8, pp. 3861-3866 (1989)). In particular, a ribozyme coding DNA sequence, designed according to the previous rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

Antisense Molecules

In another embodiment, activity of a target RNA (preferable mRNA) species, specifically its rate of translation, can be controllably inhibited by the controllable application of antisense nucleic acids. Application at high levels results in a saturating inhibition. An “antisense” nucleic acid as used herein refers to a nucleic acid capable of hybridizing to a sequence-specific (e.g., non-poly A) portion of the target RNA, for example, its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety or phosphate backbone. The oligonucleotide may include other appending groups such as peptides, or agents facilitating transport across the cell membrane (see, e.g., Letsinger et al., Proc. Natl. Acad. Sci. USA, Vol. 86, pp. 6553-6556 (1989); Lemaitre et al., Proc. Natl. Acad. Sci. USA, Vol. 84, pp. 648-652 (1987); PCT Publication No. WO 88/09810, published Dec. 15, 1988), hybridization-triggered cleavage agents (see, e.g., Krol et al., BioTechniques, Vol. 6, pp. 958-976 (1988)) or intercalating agents (see, e.g., Zon, Pharm. Res., Vol. 5, pp. 539-549 (1988)).

In a preferred aspect of the invention, an antisense oligonucleotide is provided, preferably as single-stranded DNA. The oligonucleotide may be modified at any position on its structure with constituents generally known in the art.

Typical antisense approaches involve the preparation of oligonucleotides, either DNA or RNA that are complementary to the encoded mRNA of the gene. The antisense oligonucleotides will hybridize to the encoded mRNA of the gene and prevent translation. The capacity of the antisense nucleotide sequence to hybridize with the desired gene will depend on the degree of complementarity and the length of the antisense nucleotide sequence. Typically, as the length of the hybridizing nucleic acid increases, the more base mismatches with an RNA it may contain and still form a stable duplex or triplex. One skilled in the art can determine a tolerable degree of mismatch by use of conventional procedures to determine the melting point of the hybridized complexes.

Antisense oligonucleotides are preferably designed to be complementary to the 5′ end of the mRNA, e.g., the untranslated sequence up to, and including, the regions complementary to the mRNA initiation site, i.e., AUG. However, olionucleotide sequences that are complementary to the 3′ untranslated sequence of mRNA have also been shown to be effective at inhibiting translation of mRNAs as described, e.g., in Wagner, Nature, Vol. 372, p. 333 (1994). While antisense oligonucleotides can be designed to be complementary to the mRNA coding regions, such oligonucleotides are less efficient inhibitors of translation.

The antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine,

5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of: a phosphorothioate, a phosphorodithioate, a phosphoramidothioate, a phosphoramidate, a phosphordiamidate, a methylphosphonate, an alkyl phosphotriester and a formacetal or analog thereof.

In yet another embodiment, the oligonucleotide is a 2-a-anomeric oligonucleotide. An a-anomeric oligonucleotide forms specific double-stranded hybrids with complementary RNA in which, contrary to the usual B-units, the strands run parallel to each other (Gautier et al., Nucl. Acids Res., Vol. 15, pp. 6625-6641 (1987)).

The oligonucleotide may be conjugated to another molecule, e.g., a peptide, hybridization triggered cross-linking agent, transport agent, hybridization-triggered cleavage agent, etc.

The antisense nucleic acids of the invention comprise a sequence complementary to at least a portion of a target RNA species. However, absolute complementarity, although preferred, is not required. A sequence “complementary to at least a portion of an RNA”, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibiting translation of the target RNA can be determined by standard assay techniques.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al., Nucl. Acids Res., Vol. 16, p. 3209 (1988), methylphosphonate oligonucleotides can be prepared by use of controlled pore glass polymer supports (see Sarin et al., Proc. Natl. Acad. Sci. USA, Vol. 85, pp. 7448-7451 (1988)), etc. In another embodiment, the oligonucleotide is a 2′-O-methylribonucleotide (Inoue et al., Nucl. Acids Res., Vol. 15, pp. 6131-6148 (1987)), or a chimeric RNA-DNA analog (Inoue et al., FEBS Lett., Vol. 215, pp. 327-330 (1987)).

The synthesized antisense oligonucleotides can then be administered to a cell in a controlled or saturating manner. For example, the antisense oligonucleotides can be placed in the growth environment of the cell at controlled levels where they may be taken up by the cell. The uptake of the antisense oligonucleotides can be assisted by use of methods well-known in the art.

When introduced into a host cell, antisense nucleotide sequences specifically hybridize with the cellular mRNA and/or genomic DNA corresponding to the gene(s) so as to inhibit expression of the encoded protein, e.g., by inhibiting transcription and/or translation within the cell.

The isolated nucleic acid molecule comprising the antisense nucleotide sequence can be delivered, e.g., as an expression vector, which when transcribed in the cell, produces RNA which is complementary to at least a unique portion of the encoded mRNA of the gene(s). Alternatively, the isolated nucleic acid molecule comprising the antisense nucleotide sequence is an oligonucleotide probe which is prepared ex vivo and, which when introduced into the cell, results in inhibiting expression of the encoded protein by hybridizing with the mRNA and/or genomic sequences of the gene(s).

Preferably, the oligonucleotide contains artificial internucleotide linkages, which render the antisense molecule resistant to exonucleases and endonucleases, and thus are stable in the cell. Examples of modified nucleic acid molecules for use as antisense nucleotide sequences are phosphoramidate, phosporothioate and methylphosphonate analogs of DNA as described, e.g., in U.S. Pat. Nos. 5,176,996; 5,264,564; and 5,256,775. General approaches to preparing oligomers useful in antisense therapy are described, e.g., in Van der Krol., BioTechniques, Vol. 6, pp. 958-976 (1988); and Stein et al., Cancer Res., Vol. 48, pp. 2659-2668 (1988).

Antisense Molecules Expressed Intracellularly

As discussed above, antisense nucleotides can be delivered to cells which express the described genes in vivo by various techniques, e.g., injection directly into the breast tissue site, entrapping the antisense nucleotide in a liposome, by administering modified antisense nucleotides which are targeted to the breast cells by linking the antisense nucleotides to peptides or antibodies that specifically bind receptors or antigens expressed on the cell surface.

However, with the above-mentioned delivery methods, it may be difficult to attain intracellular concentrations sufficient to inhibit translation of endogenous mRNA. Accordingly, in an alternative embodiment, the nucleic acid comprising an antisense nucleotide sequence is placed under the transcriptional control of a promoter, i.e., a DNA sequence which is required to initiate transcription of the specific genes, to form an expression construct. The antisense nucleic acids of the invention are controllably expressed intracellularly by transcription from an exogenous sequence. If the expression is controlled to be at a high level, a saturating perturbation or modification results. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include the Tet promoter. Other usable promoters for mammalian cells include, but are not limited to, the SV40 early promoter region (see Bernoist and Chambon, Nature, Vol. 290, pp. 304-310 (1981)), the promoter contained in the 3′ long terminal repeat of Rous sarcoma virus (Yamamoto et al., Cell, Vol. 22, pp. 787-797 (1980)), the herpes thymidine kinase promoter (Wagner et al., Proc. Natl. Acad. Sci. USA, Vol. 78, pp. 1441-1445 (1981)), the regulatory sequences of the metallothionein gene (Brinster et al., Nature, Vol. 296, pp. 39-42 (1982)), etc.

Therefore, antisense nucleic acids can be routinely designed to target virtually any mRNA sequence, and a cell can be routinely transformed with or exposed to nucleic acids coding for such antisense sequences such that an effective and controllable or saturating amount of the antisense nucleic acid is expressed. Accordingly the translation of virtually any RNA species in a cell can be modified or perturbed.

Double-Stranded RNA

Double-stranded RNA, i.e., sense-antisense RNA, corresponding to at least one of the disclosed genes, can also be utilized to interfere with expression of at least one of the disclosed genes. Interference with the function and expression of endogenous genes by double-stranded RNA has been shown in various organisms such as C. elegans as described, e.g., in Fire et al., Nature, Vol. 391, pp. :806-811 (1998).

RNA Aptamers

Finally, in a further embodiment, RNA aptamers can be introduced into or expressed in a cell. RNA aptamers are specific RNA ligands for proteins, such as for Tat and Rev RNA (Good et al., Gene Therapy, Vol. 4, pp. 45-54 (1997)) that can specifically inhibit their translation.

Methods of Modifyinq the Abundance or Activity of Expressed Protein

Methods of modifying protein abundance include, inter alia, those altering protein degradation rates and those using antibodies (which bind to proteins affecting abundance of activities of native target protein species). Methods of directly modifying protein activities include, inter alia, the use of antibodies, dominant negative mutations, specific drugs or chemical moieties.

Increasing (or decreasing) the degradation rates of a protein species decreases (or increases) the abundance of that species. Methods for increasing the degradation rate of a target protein in response to elevated temperature and/or exposure to a particular drug, which are known in the art, can be employed in this invention. For example, one such method employs a heat-inducible or drug-inducible N-terminal degron, which is an N-terminal protein fragment that exposes a degradation signal promoting rapid protein degradation at a higher temperature (e.g., 37° C.) and which is hidden to prevent rapid degradation at a lower temperature (e.g., 23° C.) (see Dohmen et al., Science, Vol. 263, pp. 1273-1276 (1994)). Such an exemplary degron is Arg-DHFR^{ts}, a variant of murine dihydrofolate reductase in which the N-terminal Val is replaced by Arg and the Pro at position 66 is replaced with Leu. According to this method, for example, a gene for a target protein, P, is replaced by standard gene targeting methods known in the art (Lodish et al., “Molecular Biology of the Cell”, W. H. Freeman and Co., NY (1995), especially chap 8) with a gene coding for the fusion protein Ub-Arg-DHFR^{ts}-P (“Ub” stands for ubiquitin). The N-terminal ubiquitin is rapidly cleaved after translation exposing the N-terminal degron. At lower temperatures, lysines internal to Arg-DHFR^{ts} are not exposed, ubiquitination of the fusion protein does not occur, degradation is slow, and active target protein levels are high. At higher temperatures (in the absence of methotrexate), lysines internal to Arg-DHFR^{ts} are exposed, ubiquitination of the fusion protein occurs, degradation is rapid, and active target protein levels are low.

This technique also permits controllable modification of degradation rates since heat activation of degradation is controllably blocked by exposure methotrexate. This method is adaptable to other N-terminal degrons that are responsive to other inducing factors, such as drugs and temperature changes. Also, one of skill in the art will appreciate that expression of antibodies binding and inhibiting a target protein can be employed as another dominant negative strategy.

Modifying Expressed Protein Activity with Small Molecule Drugs or Ligands

In addition, the activities of certain target proteins can be modified or perturbed in a controlled or a saturating manner by exposure to exogenous drugs or ligands. Since the methods of this invention are often applied to testing or confirming the usefulness of various drugs to treat cancer, drug exposure is an important method of modifying/perturbing cellular constituents, both mRNAs and expressed proteins. In a preferred embodiment, input cellular constituents are perturbed either by drug exposure or genetic manipulation (such as gene deletion or knockout) and system responses are measured by gene expression technologies (such as hybridization to gene transcript arrays, described in the following).

In a preferable case, a drug is known that interacts with only one target protein in the cell and alters the activity of only that one target protein, either increasing or decreasing the activity. Graded exposure of a cell to varying amounts of that drug thereby causes graded perturbations of network models having that target protein as an input. Saturating exposure causes saturating modification/perturbation. For example, Cyclosporin A is a very specific regulator of the calcineurin protein, acting via a complex with cyclophilin. A titration series of Cyclosporin A therefore can be used to generate any desired amount of inhibition of the calcineurin protein. Alternately, saturating exposure to Cyclosporin A will maximally inhibit the calcineurin protein.

Modifying Protein Activity With Antibodies and Antagonists

The term “antagonist” refers to a molecule which, when bound to the protein encoded by the gene, inhibits its activity. Antagonists can include, but are not limited to, peptides, proteins, carbohydrates and small molecules.

In a particularly useful embodiment, the antagonist is an antibody specific for the cell-surface protein expressed by at least one gene. Antibodies useful as therapeutics encompass the antibodies as described above. The antibody alone may act as an effector of therapy or it may recruit other cells to actually effect cell killing. The antibody may also be conjugated to a reagent such as a chemotherapeutic, radionuclide, ricin A chain, cholera toxin, pertussis toxin, etc., and serve as a target agent. Alternatively, the effector may be a lymphocyte carrying a surface molecule that interacts, either directly or indirectly, with a tumor target. Various effector cells include cytotoxic T-cells and NK-cells.

Examples of the antibody-therapeutic agent conjugates which can be used in therapy include, but are not limited to:

1) Antibodies coupled to radionuclides, such as ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho′, ¹⁷⁷Lu, ¹⁸⁶Re and ¹⁸⁸Re, and as described, e.g., in Goldenberg et al., Cancer Res., Vol. 41, pp. 4354-4360 (1981); Carrasquillo et al., Cancer Treat. Rep., Vol. 68, pp. 317-328 (1984); Zalcberg et al.; J. Natl. Cancer Inst., Vol. 72, pp. 697-704 (1984); Jones 1 et al., Int. J. Cancer, Vol. 35, pp. 715-720 (1985); Lange et al., Surgery, Vol. 98, pp. 143-150 (1985); Kaltovich et al., J. Nucl. Med., Vol. 27, pp. 897 (1986); Order et al., Int. J. Radiother. Oncol. Biol. Phys., Vol. 8, pp. 259-261 (1982); Courtenay-Luck et al., Lancet, Vol. 1, pp. 1441-1443 (1984); and Ettinger et al., Cancer Treat. Rep., Vol. 66, pp. 289-297 (1982);

2) Antibodies coupled to drugs or biological response modifiers, such as methotrexate, adriamycin and lymphokines, such as interferon as described, for, e.g., in Chabner et al., “Cancer, Principles and Practice of Oncology”, J. B. Lippincott Co., Philadelphia, Pa., Vol. 1, pp. 290-328 (1985); Oldham et al., “Principles and Practice of Oncology”, Cancer, J. B. Lippincott Co., Philadelphia, Pa., Vol. 2, pp. 2223-2245 (1985); Deguchi et al., Cancer Res., Vol. 46, pp. 43751-43755 (1986); Deguchi et al., Fed. Proc., Vol. 44, p. 1684 (1985); Embleton et al., Br. J. Cancer, Vol. 49, pp. 559-565 (1984); and Pimm et al., Cancer Immunol. Immunother., Vol. 12, pp. 125-134 (1982);

3) Antibodies coupled to toxins, as described, for example, in Uhr et al., “Monoclonal Antibodies and Cancer”, Academic Press, Inc., pp. 85-98 (1983); Vitetta et al., “Biotechnology and Bio. Frontiers”, P. H. Abelson, Ed., pp. 73-85 (1984); and Vitetta et al., Science, Vol. 219, pp. 644-650 (1983);

4) Heterofunctional antibodies, for example, antibodies coupled or combined with another antibody so that the complex binds both to the carcinoma and effector cells, e.g., killer cells such as T-cells, as described, for example, in Perez et al., J. Exper. Med., Vol. 163, pp. 166-178 (1986); and Lau et al., Proc. Natl. Acad. Sci. USA, Vol. 82, pp. 8648-8652 (1985); and

5) Native, i.e., non-conjugated or non-complexed, antibodies, as described in, for example, Herlyn et al., Proc. Natl. Acad. Sci. USA, Vol. 79, pp. 4761-4765 (1982); Schulz et al., Proc. Natl. Acad. Sci. USA, Vol. 80, pp. 5407-5411 (1983); Capone et al., Proc. Natl. Acad. Sci. USA, Vol. 80, pp. 7328-7332 (1983); Sears et al., Cancer Res., Vol. 45, pp. 5910-5913 (1985); Nepom et al., Proc. Natl. Acad. Sci. USA, Vol. 81, pp. 2864-2867 (1984); Koprowski et al., Proc. Nat. Acad. Sci. USA, Vol. 81, pp. 216-219 (1984); and Houghton et al., Proc. Natl. Acad. Sci. USA, Vol. 82, pp. 1242-1246 (1985).

Methods for coupling an antibody or fragment thereof to a therapeutic agent as described above are well known in the art and are described, e.g., in the methods provided in the references above.

Use of an Antagonist as a Therapeutic

In yet another embodiment, the antagonist useful as a therapeutic for treating breast cancer can be an inhibitor of a protein encoded by one of the disclosed genes. For example, the activity of the membrane-bound serine protease hepsin can be inhibited by utilizing specific serine protease inhibitors, which, in turn, would block the growth of malignant breast cells with minimal system toxicity. Such serine-protease inhibitors are well-known in the art. For example, arotinin is a serine protease inhibitor approved for reducing blood loss and transfusion requirements in cardiopulmonary bypass, inhibits kallikrein and plasmin, resulting in suppression of multiple systems involved in the inflammatory response (see Ann. Thorac. Surg., Vol. 71, No. 2, pp. 745-754 (2001)).

Maspin (mammary serpin) is a novel serine protease inhibitor related to the serpin family with a tumor-suppressing function in breast cancer (see Acta. Oncol., Vol. 39, No. 8, pp. 931-934 (2000)).

Thrombin and factor Xa (fXa) are the only serine proteases for which small, potent, selective, noncovalent inhibitors have been developed, which are ultimately intended as drug development candidates (in this case as anticoagulants) (see Med. Res. Rev., Vol. 19, No. 2, pp. 179-197 (1999)).

Target protein activities can also be decreased by (neutralizing) antibodies. By providing for controlled or saturating exposure to such antibodies, protein abundance/activities can be modified or perturbed in a controlled or saturating manner. For example, antibodies to suitable epitopes on protein surfaces may decrease the abundance, and thereby indirectly decrease the activity, of the wild-type active form of a target protein by aggregating active forms into complexes with less or minimal activity as compared to the wild-type unaggregated wild-type form. Alternately, antibodies may directly decrease protein activity by, e.g., interacting directly with active sites or by blocking access of substrates to active sites. Conversely, in certain cases, (activating) antibodies may also interact with proteins and their active sites to increase resulting activity. In either case, antibodies (of the various types to be described) can be raised against specific protein species (by the methods to be described) and their effects screened. The effects of the antibodies can be assayed and suitable antibodies selected that raise or lower the target protein species concentration and/or activity. Such assays involve introducing antibodies into a cell (see below), and assaying the concentration of the wild-type amount or activities of the target protein by standard means (such as immunoassays) known in the art. The net activity of the wild-type form can be assayed by assay means appropriate to the known activity of the target protein.

Introduction of Antibodies into Cells

Antibodies can be introduced into cells in numerous fashions, including, for example, microinjection of antibodies into a cell (see Morgan et al., Immunology Today, Vol. 9, pp. 84-86 (1988)) or transforming hybridoma mRNA encoding a desired antibody into a cell (see Burke et al., Cell, Vol. 36, pp. 847-858 (1984)). In a further technique, recombinant antibodies can be engineering and ectopically expressed in a wide variety of non-lymphoid cell types to bind to target proteins as well as to block target protein activities (Biocca et al., Trends in Cell Biology, Vol. 5, pp. 248-252 (1995)). Expression of the antibody is preferably under control of a controllable promoter, such as the Tet promoter, or a constitutively active promoter (for production of saturating perturbations). A first step is the selection of a particular monoclonal antibody with appropriate specificity to the target protein (see below). Then sequences encoding the variable regions of the selected antibody can be cloned into various engineered antibody formats, including, for example, whole antibody, Fab fragments, Fv fragments, single chain Fv fragments (V_{H} and V_{L} regions united by a peptide linker) (“ScFv” fragments), diabodies (two associated ScFv fragments with different specificity), and so forth (Hayden et al., Current Opinion in Immunology, Vol. 9, pp. 210-212 (1997)). Intracellularly expressed antibodies of the various formats can be targeted into cellular compartments (e.g., the cytoplasm, the nucleus, the mitochondria, etc.) by expressing them as fusion's with the various known intracellular leader sequences (Bradbury et al., Antibody Engineerinq, Vol. 2, Borrebaeck, Ed., pp. 295-361, IRL Press (1995)). In particular, the ScFv format appears to be particularly suitable for cytoplasmic targeting.

The Variety of Useful Antibody Types

Antibody types include, but are not limited to, polyclonal, monoclonal, chimeric, single chain, Fab fragments and an Fab expression library. Various procedures known in the art may be used for the production of polyclonal antibodies to a target protein. For production of the antibody, various host animals can be immunized by injection with the target protein, such host animals include, but are not limited to, rabbit, mice, rats, etc. Various adjuvants can be used to increase the immunological response, depending on the host species, and include, but are not limited to, Freunds (complete and incomplete), mineral gels, such as aluminum hydroxide, surface active substances such as lysolecithin, pluronic polyols, polyanions, peptides, oil emulsions, dinitrophenol, and potentially useful human adjuvants such as bacillus Calmette-Guerin (BCG) and corynebacterium parvum.

Monoclonal Antibodies

For preparation of monoclonal antibodies directed towards a target protein, any technique that provides for the production of antibody molecules by continuous cell lines in culture may be used. Such techniques include, but are not restricted to, the hybridoma technique originally developed by Kohler and Milstein, Nature, Vol. 256, pp. 495-497 (1975)), the trioma technique, the human B-cell hybridoma technique (See Kozbor et al., Immunology Today, Vol. 4, p. 72 (1983)), and the EBV hybridoma technique to produce human monoclonal antibodies (Cole et al., “Monoclonal Antibodies and Cancer Therapy”, Alan R. Liss, Inc., pp. 77-96 (1985)). In an additional embodiment of the invention, monoclonal antibodies can be produced in germ-free animals utilizing recent technology (PCT/US90/02545). According to the invention, human antibodies may be used and can be obtained by using human hybridomas (see Cote et al., Proc. Natl. Acad. Sci. USA, Vol. 80, pp. 2026-2030 (1983)), or by transforming human B cells with EBV virus in vitro (see Cole et al., “Monoclonal Antibodies and Cancer Therapy”, Alan R. Liss, Inc., pp. 77-96 (1985)). In fact, according to the invention, techniques developed for the production of “chimeric antibodies” (see Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985)) by splicing the genes from a mouse antibody molecule specific for the target protein together with genes from a human antibody molecule of appropriate biological activity can be used; such antibodies are within the scope of this invention.

Additionally, where monoclonal antibodies are advantageous, they can be alternatively selected from large antibody libraries using the techniques of phage display (see Marks et al., J. Biol. Chem., Vol. 267, pp. 16007-16010 (1992)). Using this technique, libraries of up to 10¹² different antibodies have been expressed on the surface of fd filamentous phage, creating a “single pot” in vitro immune system of antibodies available for the selection of monoclonal antibodies (see Griffiths et al., EMBO J., Vol. 13, pp. 3245-3260 (1994)). Selection of antibodies from such libraries can be done by techniques known in the art, including contacting the phage to immobilized target protein, selecting and cloning phage bound to the target, and subcloning the sequences encoding the antibody variable regions into an appropriate vector expressing a desired antibody format.

According to the invention, techniques described for the production of single chain antibodies (U.S. Pat. No. 4,946,778) can be adapted to produce single chain antibodies specific to the target protein. An additional embodiment of the invention utilizes the techniques described for the construction of Fab expression libraries (see Huse et al., Science, Vol. 246, pp. 1275-1281 (1989)) to allow rapid and easy identification of monoclonal Fab fragments with the desired specificity for the target protein.

Antibody fragments that contain the idiotypes of the target protein can be generated by techniques known in the art. For example, such fragments include, but are not limited to: the F(ab′)₂ fragment which can be produced by pepsin digestion of the antibody molecule; the Fab′ fragments that can be generated by reducing the disulfide bridges of the F(ab′)₂ fragment, the Fab fragments that can be generated by treating the antibody molecule with papain and a reducing agent, and Fv fragments.

In the production of antibodies, screening for the desired antibody can be accomplished by techniques known in the art, e.g., ELISA. To select antibodies specific to a target protein, one may assay generated hybridomas or a phage display antibody library for an antibody that binds to the target protein.

Other Methods of Modifying Protein Activities

Dominant negative mutations are mutations to endogenous genes or mutant exogenous genes that when expressed in a cell disrupt the activity of a targeted protein species. Depending on the structure and activity of the targeted protein, general rules exist that guide the selection of an appropriate strategy for constructing dominant negative mutations that disrupt activity of that target (see Hershkowitz, Nature, Vol. 329, pp. 219-222 (1987)). In the case of active monomeric forms, over expression of an inactive form can cause competition for natural substrates or ligands sufficient to significantly reduce net activity of the target protein. Such over expression can be achieved by, for example, associating a promoter, preferably a controllable or inducible promoter, or also a constitutively expressed promoter, of increased activity with the mutant gene. Alternatively, changes to active site residues can be made so that a virtually irreversible association occurs with the target ligand. Such can be achieved with certain tyrosine kinases by careful replacement of active site serine residues (see Perlmutter et al., Current Opinion in Immunology, Vol. 8, pp. 285-290 (1996)).

In the case of active multimeric forms, several strategies can guide selection of a dominant negative mutant. Multimeric activity can be decreased in a controlled or saturating manner by expression of genes coding exogenous protein fragments that bind to multimeric association domains and prevent multimer formation. Alternatively, controllable or saturating over expression of an inactive protein unit of a particular type can tie up wild-type active units in inactive multimers, and thereby decrease multimeric activity (see Nocka et al., EMBO J., Vol. 9, pp. 1805-1813 (1990)). For example, in the case of dimeric DNA binding proteins, the DNA binding domain can be deleted from the DNA binding unit, or the activation domain deleted from the activation unit. Also, in this case, the DNA binding domain unit can be expressed without the domain causing association with the activation unit. Thereby, DNA binding sites are tied up without any possible activation of expression. In the case where a particular type of unit normally undergoes a conformational change during activity, expression of a rigid unit can inactivate resultant complexes. For a further example, proteins involved in cellular mechanisms, such as cellular motility, the mitotic process, cellular architecture, and so forth, are typically composed of associations of many subunits of a few types. These structures are often highly sensitive to disruption by inclusion of a few monomeric units with structural defects. Such mutant monomers disrupt the relevant protein activities and can be expressed in a cell in a controlled or saturating manner.

In addition to dominant negative mutations, mutant target proteins that are sensitive to temperature (or other exogenous factors) can be found by mutagenesis and screening procedures that are well-known in the art.

Treatment Modalities

In the case of treatment with an antisense nucleotide, the method comprises administering a therapeutically effective amount of an isolated nucleic acid molecule comprising an antisense nucleotide sequence derived from at least one gene identified in Tables 1, 2, 3 or 4, wherein the antisense nucleotide has the ability to change the transcription/translation of the at least one gene. The term “isolated” nucleic acid molecule means that the nucleic acid molecule is removed from its original environment (e.g., the natural environment if it is naturally occurring). For example, a naturally occurring nucleic acid molecule is not isolated, but the same nucleic acid molecule, separated from some or all of the co-existing materials in the natural system, is isolated, even if subsequently reintroduced into the natural system. Such nucleic acid molecules could be part of a vector or part of a composition and still be isolated, in that such vector or composition is not part of its natural environment.

With respect to treatment with a ribozyme or double-stranded RNA molecule, the method comprises administering a therapeutically effective amount of a nucleotide sequence encoding a ribozyme, or a double-stranded RNA molecule, wherein the nucleotide sequence encoding the ribozyme/double-stranded RNA molecule has the ability to change the transcription/translation of the at least one gene.

In the case of treatment with an antagonist, the method comprises administering to a subject a therapeutically effective amount of an antagonist that inhibits or activates a protein encoded by at least one gene identified in Tables 1, 2, 3 or 4.

A “therapeutically effective amount” of an isolated nucleic acid molecule comprising an antisense nucleotide, nucleotide sequence encoding a ribozyme, double-stranded RNA, or antagonist, refers to a sufficient amount of one of these therapeutic agents to treat breast cancer (e.g., to limit breast tumor growth or to slow or block tumor metastasis). The determination of a therapeutically effective amount is well within the capability of those skilled in the art. For any therapeutic, the therapeutically effective dose can be estimated initially either in cell culture assays, e.g., of neoplastic cells, or in animal models, usually mice, rabbits, dogs or pigs. The animal model may also be used to determine the appropriate concentration range and route of administration. Such information can then be used to determine useful doses and routes for administration in humans.

Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutically effects is the therapeutic index, and it can be expressed as the ratio LD₅₀/ED₅₀. Antisense nucleotides, ribozymes, double-stranded RNAs and antagonists that exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range, depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight and gender of the subject, diet, time and frequency of administration, drug combination(s), reaction sensitivities, and tolerance/response to therapy.

Normal dosage amounts may vary form 0.1-100,000 micrograms, up to a total dosage of about 1 g, depending upon the route of administration. Guidance as to particular dosages and methods of delivery is provided in the literature and generally available to practitioners in the art. Those skilled in the art will employ different formulations for nucleotides than for antagonists.

For therapeutic applications, the antisense nucleotides, nucleotide sequences encoding ribozymes, double-stranded RNAs (whether entrapped in a liposome or contained in a viral vector) and antibodies are preferably administered as pharmaceutical compositions containing the therapeutic agent in combination with one or more pharmaceutically acceptable carriers. The compositions may be administered alone or in combination with at least one other agent, such as stabilizing compound, which may be administered in any sterile, biocompatible pharmaceutical carrier, including, but not limited to, saline, buffered saline, dextrose and water. The compositions may be administered to a patient alone or in combination with other agents, drugs or hormones.

The pharmaceutical compositions may be administered by an number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual or rectal means. In addition to the active ingredient, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's “Pharmaceutical Sciences”, Maack Publishing Co., Easton, Pa.

Pharmaceutical compositions for oral administration can be formulated using pharmaceutically acceptable carriers well-known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients re carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins, such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations, which can be used orally, include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated m aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycatonic amino polymers may also be used for delivery. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with many acids, including, but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder that may contain any or all of the following: 1-50 mM histidine, 0.1-2% sucrose and 2-7% mannitol, at a pH range of 4.5-5.5, that is combined with buffer prior to use.

After pharmaceutical compositions have been prepared, they can be placed in an appropriate container and labeled for treatment of an indicated condition. For administration of the antisense nucleotide or antagonist, such labeling would include amount, frequency, and method of administration. Those skilled in the art will employ different formulations for antisense nucleotides than for antagonists, e.g., antibodies or inhibitors. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

In another aspect, the treatment of a subject with a therapeutic agent such as those described, above, can be monitored by detecting the level of expression of mRNA or protein encoded by at least one of the disclosed genes, or the activity of the protein encoded by at least one of the disclosed genes. These measurements will indicate whether the treatment is effective or whether it should be adjusted or optimized. Accordingly, one or more of the genes describe herein can be used as a marker for the efficacy of a drug during clinical trials.

In a particularly useful embodiment, a method for monitoring the efficacy of a treatment of a subject having breast cancer or at risk of developing breast cancer with an agent (e.g., an antagonist, protein, nucleic acid, small molecule, or other therapeutic agent or candidate agent identified by the screening assays described herein) is provided comprising:

a) Obtaining a pre-administration sample from a subject prior to administration of the agent;

b) Detecting the level of expression of mRNA or protein encoded by the at least one gene, or activity of the protein encoded by the at least one gene in the pre-administration sample;

c) Obtaining one or more post-administration samples from the subject;

d) Detecting the level of expression of mRNA or protein encoded by the at least one gene, or activity of the protein encoded by the at least one gene in the post-administration sample or samples;

e) Comparing the level of expression of mRNA or protein encoded by the at least one gene, or activity of the protein encoded by the at least one gene in the pre-administration sample with the level of expression of mRNA or protein encoded by the at least one gene, or activity of the protein encoded by the at least one gene in the post-administration sample or samples; and

f) Adjusting the of the agent accordingly.

For example, increased administration of the agent may be desirable to change the level of expression or activity of the at least one gene to higher or lower levels than detected, i.e., to increase the effectiveness of the agent. Alternatively, decreased administration of the agent may be desirable to change expression of the at least one gene to higher or lower levels than detected, i.e., to decrease the effectiveness of the agent.

In another aspect, a method for inhibiting the proliferation of breast cancer tissue in a subject is provided which utilizes a therapeutic agent as described above, e.g., an antisense nucleotide, a ribozyme, a double-stranded RNA, and an antagonist such as an antibody. With respect to inhibition of proliferation of breast cancer tissue utilizing an antisense nucleotide, the method comprises administering to the subject a therapeutically effective amount of an isolated nucleic acid molecule comprising an antisense nucleotide sequence derived from at least one gene identified in Tables 1, 2, 3 or 4, wherein the antisense nucleotide has the ability to change the transcription/translation of the at least one gene.

With respect to inhibition of proliferation of breast cancer tissue utilizing a ribozyme, such a method comprises administering to the subject a therapeutically effective amount of a nucleotide sequence encoding the ribozyme, which has the ability to change the transcription/translation of at least one gene identified in Tables 1, 2, 3 or 4.

With respect to inhibition of proliferation of breast cancer tissue utilizing a double-stranded RNA, the method comprises administering to the subject a therapeutically effective amount of a double-stranded RNA corresponding to at least one gene identified in Tables 1, 2, 3 or 4, wherein the double-stranded RNA has the ability to change the transcription/translation of the at least one gene.

With respect to inhibition of proliferation of breast cancer tissue utilizing an antagonist, the method comprises administering to the subject a therapeutically effective amount of an antagonist that results in inhibition or activation of a protein encoded by at least one gene identified in Tables 1, 2, 3 or 4.

In the context of inhibiting proliferation of a breast cancer tissue, a “therapeutically effective amount” of an isolated nucleic acid molecule comprising an antisense nucleotide, a nucleotide sequence encoding a ribozyme, a double-stranded RNA, or antagonist, refers to a sufficient amount of one of these therapeutic agents to inhibit proliferation of a breast cancer tissue (e.g., to inhibit or stabilize cellular growth of the breast cancer tissue) and can be determined as described above.

The Use of Viral Vectors

In another aspect, a viral vector is provided which comprises a promoter of a gene selected from the group consisting of at least one of the genes identified in Tables 1, 2, 3 or 4, operably linked to the coding region of a gene that is essential for replication of the vector, wherein the vector is adapted to replicate upon transfection into a breast cell.

Such vectors are able to selectively replicate in a breast tissue, but not in non-breast tissue. The replication is conditioned upon the presence in breast tissue, and not in non-breast tissue, of positive transcription factors that activates the promoter of the disclosed genes. It can also occur by the absence of transcription inhibiting factors that normally occur in non-breast tissue and prevent transcription as a result of the promoter. Accordingly, when transcription occurs, it proceeds into the gene essential for replication such that in the breast tissue, but not in non-breast tissue, replication of the vector and its attendant functions occur. With this vector, a diseased breast tissue, e.g., breast tumor, can be selectively treated, with minimal systemic toxicity.

In one embodiment, the viral vector is an adenoviral vector, which includes a coding region of a gene essential for replication of the vector, wherein the coding region is selected from the group consisting of E1, E1, E2 and E4 coding regions. Methods for making such vectors are well-known to the person of ordinary skill in the art as described, e.g., in Sambrook et al., “Molecular Cloning: A Laboratory Manual”, Cold Spring Harbor, N.Y. (1989).

In a further embodiment, the vector encodes a heterologous gene product that is expressed from the vector in the breast cells. The heterologous gene product provides for the inhibition, prevention or destruction of the growth of the diseased breast tissue, e.g., breast tumor.

The gene product can be RNA, e.g., antisense RNA or ribozyme, or proteins such as a cytokine, e.g., interleukin, interferon, or toxins such as diphtheria toxin, pseudomonas toxin, etc. The heterologous gene product can also be a negative selective marker such as cytosine deaminase. Such negative selective markers can interact with other agents to prevent, inhibit or destroy the growth of the diseased breast cells.

The vector of the present invention can be transfected into a helper cell line for viral replication and to generate infectious viral particles. Alternatively, transfection of the vector into a breast cell can take place by electroporation, calcium phosphate precipitation, microinjection, or through proteoliposomes. Methods for preparing tissue-specific replication vectors and their use in the treatment of tumor cells and other types of abnormal cells which are harmful or otherwise unwanted in vivo in a subject are described in detail, e.g., in U.S. Pat. No. 5,998,205.

The Detection of Nucleic Acids and Proteins as Markers

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from breast cells (see, e.g., Ausubel, et al., Ed., “Current Protocols in Molecular Biology”, John Wiley & Sons, NY (1987-1999). Additionally, large numbers of tissue samples can readily be processed using techniques well-known to those of skill in the art, such as, for example, the single-step RNA isolation process of Chomczynski, U.S. Pat. No. 4,843,155 (1989).

The isolated mRNA can be used in hybridization or amplification assays that include, but are not limited to, Southern or Northern analyses, polymerase chain reaction analyses and probe arrays. One preferred diagnostic method for the detection of mRNA levels involve contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7,15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example, by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by rtPCR (the experimental embodiment set forth in Mullis, U.S. Pat. No. 4,683,202 (1987); ligase chain reaction, Barany, Proc. Natl. Acad. Sci. USA, Vol. 88, pp. 189-193 (1991); self-sustained sequence replication, Guatelli et al., Proc. Natl. Acad. Sci. USA, Vol. 87, pp. 1874-1878 (1990); transcriptional amplification system, Kwoh et al., Proc. Natl. Acad. Sci. USA, Vol. 86, pp. 1173-1177 (1989); Q-Beta Replicase, Lizardi et al., Bio/Technology, Vol. 6, p. 1197 (1988); rolling circle replication, Lizardi et al., U.S. Pat. No. 5,854,033 (1988); or any other nucleic acid amplification method, followed by the detection of the amplified molecules using techniques well-known to those of skill in the art. These detection schemes are especially useful for the detection of the nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10-30 nucleotides in length and flank a region from about 50-200 nucleotides in length. Under appropriate conditions and with appropriate reagents, such primers permit the amplification of a nucleic acid molecule comprising the nucleotide sequence flanked by the primers.

For in situ methods, mRNA does not need to be isolated form the breast cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-breast cancer sample, or between samples from different sources.

Alternatively, the expression level can be provided as a relatively expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from breast cancer or from non-breast cancer cells of breast tissue. The choice of the cell source is dependent on the use of the relative expression level. Using expression found in normal tissues as a mean expression score aids in validating whether the marker assayed is breast specific (versus normal cells). In addition, as more data is accumulated, the mean expression value can be revised, providing improved relative expression values based on accumulated data. Expression data from breast cells provides a means for grading the severity of the breast cancer state.

In another embodiment of the present invention, a polypeptide corresponding to a marker is detected. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F(ab′)₂ can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end labeling of a DNA probe with biotin such that it can be detected with fluorescently-labeled streptavidin.

Proteins from breast cells can be isolated using techniques that are well-known to those of skill in the art. The protein isolation methods employed can, for example, be such as those described in Harlow and Lane, “Antibodies: A Laboratory Manual”, Harlow and Lane, Cold Spring Harbor Laboratory Press, Cold Spring Harbor, N.Y. (1988).

A variety of formats can be employed to determine whether a sample contains a protein that binds to a given antibody. Examples of such formats include, but are not limited to, enzyme immunoassay (EIA); radioimmunoasay (RIA), Western blot analysis and ELISA. A skilled artisan can readily adapt known protein/antibody detection methods for use in determining whether breast cells express a marker of the present invention.

In one format, antibodies or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros and magnetite.

One skilled in the art will know many other suitable carriers for binding antibody or antigen, and will be able to adapt such support for use with the present invention. For example, protein isolated from breast cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a polypeptide or nucleic acid corresponding to a marker of the invention in a biological sample (e.g., a breast-associated body fluid, serum, plasma, lymph, cystic fluid, urine, stool, csf, acitic fluid or blood). Such kits can be used to determine if a subject is suffering from, or is at increased risk of, developing breast cancer. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: 1) a first antibody (e.g., attached to a solid support) which binds to a polypeptide corresponding to a marker or the invention; and, optionally, 2) a second, different antibody which binds to either the polypeptide or the first antibody and is conjugated to a detectable label.

For oligonucleotide-based kits, the kit can comprise, for example: 1) an oligonucleotide, e.g., a detectably labeled oligonucleotide, which hybridizes to a nucleic acid sequence encoding a polypeptide corresponding to a marker of the invention; or 2) a pair of primers useful for amplifying a nucleic acid molecule corresponding to a marker of the invention. The kit can also comprise, e.g., a buffering agent, a preservative, or a protein-stabilizing agent. The kit can further comprise components necessary for detecting the detectable label (e.g., an enzyme or a substrate). The kit can also contain a control sample or a series of control samples, which can be assayed and compared to the test sample. Each component of the kit can be enclosed within an individual container and all of the various containers can be within a single package, along with instructions for interpreting the results of the assays performed using the kit.

Monitoring Clinical Trials

Monitoring the influence of agents (e.g., drug compounds) on the level of expression of a marker of the invention can be applied not only in basic drug screening, but also in clinical trials. For example, the effectiveness of an agent to affect marker expression can be monitored in clinical trials of subjects receiving treatment for breast cancer. In a preferred embodiment, the present invention provides a method for monitoring the effectiveness of treatment of a subject with an agent (e.g., an agonist, antagonist, peptidomimetic), protein, peptide, nucleic acid, small molecule, or other drug candidate) comprising the steps of:

(i) Obtaining a pre-administration sample from a subject prior to administration of the agent;

(ii) Detecting the level of expression of one or more selected markers of the invention in the pre-administration sample;

(iii) Obtaining one or more post-administration samples from the subject;

(iv) Detecting the level of expression of the marker(s) in the post-administration samples;

(v) Comparing the level of expression of the marker(s) in the pre-administration sample with the level of expression of the marker(s) in the post-administration sample or samples; and

(vi) Altering the administration of the agent to the subject accordingly.

For example, increased administration of the agent can be desirable to increase expression of the marker(s) to higher levels than detected, i.e., to increase the effectiveness of the agent. Alternatively, decreased administration of the agent can be desirable to decrease the effectiveness of the agent.

Experimental Protocol

Subtracted Libraries and Transcript Profiling

Subtracted libraries are generated using a PCR-based method that allows the isolation of clones expressed at higher levels in one population of mRNA (tester) compared to another population (driver). Both tester and driver mRNA populations are converted into cDNA by reverse transcription, and then PCR amplified using the SMART™ PCR kit from Clontech. Tester and driver cDNAs are then hybridized using the PCR-Select cDNA subtraction kit form Clontech. This technique results in both subtraction and normalization, which is an equalization of copy numbers of low-abundance and high-abundance sequences. After generation of the subtractive libraries, a group of 96 or more clones from each library is tested to confirm differential expression by reverse Southern hybridization.

For the markers of the invention identified through the above-described subtractive library hybridization technique, the “tester” source for the subtracted libraries was comprised of cDNA generated from either tissue samples from three types of breast cancer (obtained from human patients), or from breast cancer cell lines. The “driver” source for the subtracted libraries was comprised of cDNA generated from non-cancerous breast tissue cells.

For transcript profiling, nylon arrays are prepared by spotting purified PCR product onto a nylon membrane using a robotic gridding system linked to a sample database. Several thousand clones are spotted on each nylon filter.

RNA or DNA from clinical samples (tumor and normal) and cell lines are used for hybridization against the nylon arrays. The RNA or DNA is labeled utilizing an in vitro reverse transcription reaction that contains a radiolabeled nucleotide that is incorporated during the reaction. Alternatively, mRNA is converted into cDNA by reverse transcription, and then PCR amplified using the SMART PCR kit from Clontech. Hybridization experiments are carried out by combining labeled RNA or DNA samples with nylon filters in a hybridization chamber. Duplicate, independent hybridization experiments are performed to generate transcriptional profiling data (see Nature Genetics, Vol. 21 (1999)).

References Cited

All references cited herein are incorporated herein by reference in their entirety and for all purposes to the same extent as if each individual publication or patent or patent application was specifically and individually indicated to be incorporated by reference in its entirety for all purposes. In addition, all GenBank accession numbers, Unigene Cluster numbers and protein accession numbers cited herein are incorporated herein by reference in their entirety and for all purposes to the same extent as if each such number was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

We claim:

1. A method for screening a subject with breast cancer to predict the response of said breast cancer to endocrine therapy comprising:
a) detecting a level of mRNA expression corresponding to the gene NOVA1 in a breast tumor biopsy obtained from the subject to obtain a first value;
b) detecting a level of mRNA expression corresponding to the gene NOVA1 in breast tumor biopsy obtained from patients whose tumors responded to endocrine therapy to obtain a second value;
c) detecting a level of mRNA expression corresponding to the gene NOVA1 in breast tumor biopsy obtained from patients whose tumor did not respond to endocrine therapy to obtain a third value; and
d) comparing the first value with the second and third values wherein a first value similar to the second value and greater than the third predicts that the subject's tumor will respond to endocrine therapy; and wherein a first value smaller than the second value and similar to the third is indicative that the subject would not respond to endocrine therapy.

2. A method for screening a subject with breast cancer to predict the response of said breast cancer to endocrine therapy comprising:
a) detecting a level of mRNA expression corresponding to the gene IGHG3 in a breast tumor biopsy obtained from the subject to obtain a first value;
b) detecting a level of mRNA expression corresponding to the gene IGHG3 in breast tumor biopsy obtained from patients whose tumors responded to endocrine therapy to obtain a second value;
c) detecting a level of mRNA expression corresponding to the gene IGHG3 breast tumor biopsy obtained from patients whose tumor did not respond to endocrine therapy to obtain a third value; and
d) comparing the first value with the second and third values wherein a first value similar to the second value and greater than the third predicts that the subject's tumor will respond to endocrine therapy; and wherein a first value smaller than the second value and similar to the third is indicative that the subject would not respond to endocrine therapy.

3. A method for screening a subject with breast cancer to predict the response of said breast cancer to endocrine therapy comprising:
a) detecting a level of mRNA expression corresponding to at least one gene identified in Table 3 in a breast tumor biopsy obtained from the subject to obtain a first value;
b) detecting a level of mRNA expression corresponding to the at least one gene identified in (a) in breast tumor biopsy obtained from patients whose tumors responded to endocrine therapy to obtain a second value;
c) detecting a level of mRNA expression corresponding to the at least one gene identified in (a) in a breast tumor biopsy obtained from patient whose tumor did not respond to endocrine therapy to obtain a third value; and
d) comparing the first value with the second and third values wherein a first value similar to the second value and greater than the third predicts that the subject's tumor will respond to endocrine therapy; and wherein a first value smaller than the second value and similar to the third is indicative that the subject would not respond to endocrine therapy.

4. A method for screening a subject with breast cancer to predict response of said breast cancer to endocrine therapy comprising:
a) detecting a level of mRNA expression corresponding to at least one gene identified in table 4 in a breast tumor biopsy obtained from the subject to obtain a first value;
b) detecting a level of mRNA expression corresponding to the at least one gene identified in (a) in a breast tumor biopsy obtained from patients whose tumors responded to endocrine therapy to obtain a second value;
c) detecting a level of mRNA expression corresponding to the at least one gene identified in (a) in a breast tumor biopsy obtained from a patient whose tumor did not respond to endocrine therapy to obtain a third value, and
d) comparing the first value with the second and third values wherein a first value similar to the second value and lower than the third predicts that the subject's tumor will respond to endocrine therapy; and wherein a first value similar to the third value and greater than the second predicts that the subject's tumor will not respond to endocrine therapy.

5. A method of treating breast cancer in a subject in need of such treatment comprising of administering to the subject a compound that modulates the synthesis, expression or activity of one or more of the genes or gene products of the genes shown in Tables 1, 2, 3 or 4 so that at least one symptom of the breast cancer is ameliorated.

6. The method of claim 5, wherein the genes are selected from the group consisting of; sodium channel, nonvoltage-gated 1 alpha (SCNN1A); serine or cysteine proteinase inhibitor, lade A member 3 (SERPINA3); N-acylsphingosine amidohydrolase (ASAH); lipocalin 1 (LCN1); transforming growth factor-beta type III receptor (TGFBR3); glutamate receptor precursor 2 (GRIA2) and cytochrome P450, subfamily IIB (phenobarbital-inducible) CYP2B), AZGP1, NOVA1 or IGHG3.

7. The method of claim 5, wherein the gene products are selected from the group consisting of the proteins expressed by the genes; sodium channel, nonvoltage-gated 1 alpha (SCNN1A); serine or cysteine proteinase inhibitor, lade A member 3 (SERPINA3); N-acylsphingosine amidohydrolase (ASAH); lipocalin 1 (LCN1); transforming growth factor-beta type III receptor (TGFBR3); glutamate receptor precursor 2 (GRIA2) and cytochrome P450, subfamily IIB (phenobarbital-inducible) CYP2B), AZGP1, NOVA1 or IGHG3.

8. A method to determine whether a breast tumor is responsive to endocrine based therapy comprising:
a) detecting the level of expression of mRNA corresponding to at least one gene identified in Tables 1, 2, 3 or 4 in a sample of breast tumor tissue to provide a first value;
b) detecting the level of expression of mRNA corresponding to the at least one gene identified in Tables 1, 2, 3 or 4 in a sample of breast tissue obtained from a disease-free subject to provide a second value; and
c) comparing the first value with the second value, wherein a greater first value relative to the second value is indicative of the subject having a breast tumor which will respond to endocrine based therapy.

9. A method of determining whether a breast carcinoma in a subject will respond to endocrine based therapy comprising:
a) detecting the level of expression of the gene expression product of the NOVA1 gene in a patient sample from the subject to obtain a first value;
b) detecting the level of expression of the gene expression product of the NOVA1 gene in patient samples obtained from patients whose tumors responded to endocrine therapy to obtain a second value;
c) detecting the level of expression of the gene expression product of the NOVA1 gene in patient samples obtained from patients whose tumors did not respond to endocrine therapy to obtain a third value; and
d) comparing the first value with the second and third values wherein a first value similar to the second value and greater than the third is an indication that the subject's tumor will respond to endocrine therapy; and wherein a first value smaller than the second value and similar to the third is indicative that the subject's tumor will not respond to endocrine therapy.

10. The method of claim 9, wherein the level of expression of the gene product of the IGHG3 gene is detected instead of the NOVA1 gene.

11. The method of claims 9 or 10, wherein the patient sample is a breast-associated body sample, selected from the group consisting of; a breast biopsy, blood, serum, plasma, lymph, ascitic fluid, cystic fluid, urine, CSF, a breast exudate or a nipple aspirate.

12. The method of claims 9, 10 or 11 wherein the level of expression of the gene expression is assessed by detecting the presence of a protein corresponding to the gene expression product.

13. The method of claim 12, wherein the presence of the protein is detected using a reagent which specifically binds with the protein.

14. The method of claim 13, wherein the reagent is selected from the group consisting of an antibody, an antibody derivative, and an antibody fragment.

15. A test for use in determining whether a breast carcinoma in a patient will respond to endocrine based therapy comprising the reagent of claim 13 or 14 in a container suitable for contacting the breast-associated body fluid.

16. The test of claim 15, wherein the reagent comprises an antibody, and wherein said antibody specifically binds with a protein corresponding to the gene expression product of claim 12.

17. A method of treating breast cancer in a subject comprising administering to said subject a compound that modulates the synthesis, expression or activity of one or more of the genes or gene expression products of the group of genes comprising those identified in Tables 1, 2, 3 or 4, so that at least one symptom of breast cancer is ameliorated.

18. The method of claim 17, wherein the compound is selected from the group consisting of an antisense molecule, double-stranded RNA, a ribozyme, a small molecule compound, an antibody or a fragment of an antibody.

19. A method for monitoring the progression of breast cancer in a subject having, or at risk of having, breast cancer comprising measuring a level of expression of mRNA corresponding to at least one of the group of genes comprising those identified in Tables 1, 2, 3 or 4 over time in a sample of bodily fluid or breast tissue obtained from the subject, wherein an increase in the level of expression of mRNA of the at least one gene over time is indicative of the progression of the breast cancer in the subject.

20. The method in claim 19, wherein the at least one gene identified in Tables 1, 2, 3 or 4 is selected from the group consisting of TFF1, TFF3, SERPINA3, PIP, MGP, TGFRB3 and AZGP1.

21. The method of claim 19, wherein the level of expression of mRNA is detected by techniques selected from the group consisting of Northern blot analysis, reverse transcription PCR and real time quantitative PCR.

22. A method for monitoring the progression of breast cancer in a subject having, or at risk of having, breast cancer comprising measuring a level of expression of a protein encoded by at least one gene identified in Tables 1, 2, 3 or 4 over time in a sample of bodily fluid or breast tissue obtained from the subject, wherein an increase in the level of expression of the protein encoded by the at least one gene over time is indicative of the progression of the breast cancer in the subject.

23. The method in claim 22, wherein the at least one gene identified in Tables 1, 2, 3 or 4 is selected from the group consisting of TFF1, TFF3, SERPINA3, PIP, MGP, TGFRB3 and AZGP1.

24. A method for monitoring the progression of breast cancer in a subject having, or at risk of having, breast cancer comprising measuring a level of expression of mRNA corresponding to at least one gene selected from a group consisting of those identified in Tables 1, 2, 3 or 4; over time in a sample of bodily fluid or breast tissue obtained from the subject, wherein a change in the level of expression of mRNA of the at least one gene over time is indicative of the progression of the breast cancer in the subject.

25. A method for monitoring the progression of breast cancer in a subject having, or at risk of having, breast cancer comprising measuring a level of expression of a protein encoded by at least one gene selected from the group consisting of those genes identified in Tables 1, 2, 3 or 4, over time a sample of bodily fluid or breast tissue obtained from the subject, wherein a change in the level of expression of the protein encoded by the at least one gene over time is indicative of the progression of the breast cancer in the subject.

26. The method of claim 25, wherein the level of expression of the protein encoded by the at least one gene is detected through Western blotting by utilizing a labeled probe specific for the protein.

27. The method of claim 26, wherein the labeled probe is an antibody.

28. The method of claim 27, wherein the antibody is a monoclonal antibody.

29. A method for identifying agents for use in the treatment of breast cancer comprising of:
a) contacting a sample of a breast tissue obtained from a subject suspected of having breast cancer with a candidate agent;
b) detecting a level of expression of mRNA of at least one gene in the sample, wherein the at least one gene is selected from the group comprising those genes identified in Tables 1, 2, 3 or 4; and
c) comparing the level of expression of mRNA of the at least one gene in the sample in the presence of the candidate agent with a level of expression of mRNA of the at least one gene in the sample in the absence of the candidate agent, wherein a decreased or increased level of expression of the mRNA of the at least one gene in the sample in the presence of the candidate agent relative to the level of expression of the mRNA of the at least one gene in the sample in the absence of the candidate agent is indicative of an agent useful in the treatment of breast cancer.

30. The method of claim 29, wherein the at least one gene identified in Tables 1, 2, 3 or 4 is selected from the group consisting of TFF1, TFF3, SERPINA3, PIP, MGP, TGFRB3 and AZGP1.

31. The method of claim 29, wherein the level of expression of mRNA is detected by techniques selected from the group consisting of; Northern blot analysis, reverse transcription PCR and real time quantitative PCR.

32. The method of claim 29 wherein the agent is selected from the group consisting of small molecules and antisense polynucleotides.

33. A method for identifying agents for use in the treatment of breast cancer comprising of:
a) contacting a sample of a bodily fluid or breast tissue obtained form a subject suspected of having breast cancer with a candidate agent;
b) detecting a level of expression of a protein encoded by at least one gene in the sample, wherein the at least one gene is selected from the group comprising those genes identified in Tables 1, 2, 3 or 4;
c) comparing the level of expression of the protein encoded by the at least one gene in the sample in the presence of the candidate agent with a level of expression of the protein encoded by the at least one gene in the sample in the absence of the candidate agent, wherein a decreased or increased level of expression of the protein of the at least one gene in the sample in the presence of the candidate agent relative to the level of expression of the protein encoded by the at least one gene in the sample in the absence of the candidate agent is indicative of an agent useful in the treatment of breast cancer.

34. The method of claim 33, wherein the at least one gene identified in the group comprising those genes identified in Tables 1, 2, 3 or 4 is TFF1, TFF3, SERPINA3, PIP, MGP, TGFRB3 and AZGP1.

35. The method of claim 33, wherein the level of expression of the protein encoded by the at least one gene is detected through Western blotting by utilizing a labeled probe specific for the protein.

36. The method of claim 35, wherein the labeled probe is an antibody.

37. The method of claim 36, wherein the antibody is a monoclonal antibody.

38. A method for identifying agents for use in the treatment of breast cancer comprising:
a) contacting a sample of breast tissue obtained from a subject suspected of having breast cancer with a candidate agent;
b) detecting a level of expression of mRNA of at least one gene in the sample, wherein the gene is selected from the group consisting of those selected from the group comprising those genes identified in Tables 1, 2, 3 or 4;
c) comparing the level of expression of mRNA of the at last one gene in the sample in the presence of the candidate agent with a level of expression of mRNA of the at least one gene in the sample in the absence of the candidate agent, wherein a change in expression level of the mRNA of the at least one gene in the sample in the presence of the agent relative to the expression level of the mRNA of the at least one gene in the sample in the absence of the candidate agent is indicative of an agent useful in the treatment of breast cancer.

39. The method of claim 38 wherein the level of expression of mRNA is detected by techniques selected from the group consisting of Northern blot analysis, reverse transcription PCR and real time quantitative PCR.

40. The method of claim 41, wherein the agent is selected from the group consisting of small molecules and antisense polynucleotides.

41. A method for identifying agents for use in the treatment of breast cancer comprising:
a) contacting a sample of a bodily fluid or breast tissue obtained from a subject suspected of having a breast disorder with a candidate agent;
b) detecting a level of expression of a protein encoded by at least one gene in the sample, wherein the gene is selected from the group consisting of those genes identified in Tables 1, 2, 3 or 4;
c) comparing the level of expression of the protein encoded by the at least one gene in the sample in the presence of the candidate agent with a level of expression of the protein encoded by the at least one gene in the sample in the absence of the candidate agent, wherein a change in level of expression of the protein of the at least one gene in the sample in the presence of the candidate agent relative to the level of expression of the protein encoded by the at least one gene in the sample in the absence of the candidate agent is indicative of an agent useful in the treatment of breast cancer.

42. The method of claim 41, wherein the level of expression of the protein encoded by the at least one gene is detected through Western blotting by utilizing a labeled probe specific for the protein.

43. The method of claim 41, wherein the labeled probe is an antibody.

44. The method of claim 43, wherein the antibody is a monoclonal antibody.

45. The method of claim 41, wherein the agent is selected from the group consisting of small molecules and antisense polynucleotides.

46. A method of treating a subject having, or at risk of having, breast cancer comprising administering to the subject a therapeutically effective amount of an isolated nucleic acid molecule comprising of an antisense nucleotide sequence derived from at least one gene selected from the group consisting of the gene is selected from the group consisting of those genes identified in Tables 1, 2, 3 or 4, which has the ability to change the transcription/translation of the at least one gene.

47. The method of claim 46 wherein the at least one gene is selected from the group consisting of TFF1, TFF3, SERPINA3, PIP, MGP, TGFRB3 and AZGP1.

48. A method of treating a subject having, or at risk of having, breast cancer comprising; administering to the subject a therapeutically effective amount of an antagonist that inhibits/activates a protein encoded by at least one gene selected from the group consisting of the gene selected from the group consisting of those genes identified in Tables 1, 2, 3 or 4.

49. The method of claim 48, wherein the at least one gene is selected from the group consisting of TFF1, TFF3, SERPINA3, PIP, MGP, TGFRB3 and AZGP1.

50. The method of claim 48, wherein the antagonist is an antibody specific for the protein.

51. The method of claim 50, wherein the antibody is a monoclonal antibody.

52. The method of claim 51, wherein the monoclonal antibody is conjugated to a toxic reagent.

53. A method of treating a subject having, or at risk of having, breast cancer consisting of administering to the subject a therapeutically effective amount of an isolated nucleic acid molecule comprising of an antisense nucleotide sequence derived from at least one gene selected from the group consisting of gene selected from the group consisting of those genes identified in Tables 1, 2, 3 or 4, which has the ability to decrease/increase the transcription/translation of the at least one gene.

54. A method of treating a subject having, or at risk of having, breast cancer comprising of administering to the subject a therapeutically effective amount of an antagonist that inhibits/activates a protein encoded by at least one gene selected from the group consisting of the genes identified in Tables 1, 2, 3 or 4.

55. The method of claim 54, wherein the antagonist is an antibody specific for the protein.

56. The method of claim 55, wherein the antibody is a monoclonal antibody.

57. The method of claim 56, wherein the monoclonal antibody is conjugated to a toxic reagent.

58. A method of treating a subject having, or at risk of having, breast cancer comprising administering to the subject a therapeutically effective amount of a nucleotide sequence encoding a ribozyme, which has the ability to decrease/increase the transcription/translation of at least one gene selected from the group consisting of the genes identified in Tables 1, 2, 3 or 4.

59. A method of treating a subject having, or at risk of having, breast cancer comprising administering to the subject a therapeutically effective amount of a double-stranded RNA corresponding to at least one gene identified in claim 58, which has the ability to decrease the transcription/translation of the at least one gene.

60. A method of treating a subject having, or at risk of having, breast cancer comprising administering to the subject a therapeutically effective amount of a nucleotide sequence encoding a ribozyme, which has the ability to change the transcription/translation of at least one gene selected from the group consisting of the genes identified in Tables 1, 2, 3 or 4.

61.; A method of treating a subject having, or at risk of having, breast cancer comprising administering to the subject a therapeutically effective amount of a double-stranded RNA corresponding to at least one gene selected from the group consisting of those genes identified in Tables 1, 2, 3 or 4, which has the ability to change the transcription/translation of the at least one gene.

62. A method for monitoring the efficacy of a treatment of a subject having breast cancer, or at risk of developing breast cancer, with an agent, the method comprising:
a) obtaining a pre-administration sample from the subject prior to administration of the agent;
b) detecting a level of expression of mRNA corresponding to a gene selected from the group consisting of those genes identified in Tables 1, 2, 3 or 4;
c) obtaining one or more post-administration samples from the subject;
d) detecting a level of expression of mRNA corresponding to the at least one gene in the post-administration sample or samples;
e) comparing the level of expression of mRNA corresponding to the at least one gene in the pre-administration sample with the level of expression of mRNA corresponding to the at last one gene in the post-administration sample; and
f) adjusting the administration of the agent accordingly.

63. A method for monitoring the efficacy of a treatment of a subject having breast cancer, or at risk of developing breast cancer, with an agent, the method comprising:
a) obtaining a pre-administration sample from the subject prior to administration of the agent;
b) detecting a level of expression of protein encoded by at least one gene selected from the group consisting of those genes identified in Tables 1, 2, 3 or 4;
c) obtaining one or more post-administration samples from the subject;
d) detecting a level of expression of protein encoded by the at least one gene in the post-administration sample or samples;
e) comparing the level of expression of protein encoded by the at least one gene in the pre-administration sample with the level of expression of protein encoded by the at least one gene in the post-administration sample; and
f) adjusting the administration of the agent accordingly.

64. A method for inhibiting the proliferation of breast cancer tissue in a subject which comprises administering to the subject a therapeutically effective amount of an isolated nucleic acid molecule comprising of an antisense nucleotide sequence derived from at least one gene selected from the group consisting of those genes identified in Tables 1, 2, 3 or 4, which has the ability to change the transcription/translation of the at least one gene.

65. A method for inhibiting the proliferation of breast cancer tissue in a subject which comprises administering to the subject a therapeutically effective amount of an isolated nucleic acid molecule comprising of an antisense nucleotide sequence derived from at least one gene selected from the group consisting of those genes identified in Tables 1, 2, 3 or 4, which has the ability to change the transcription/translation of the at least one gene.

66. A method for inhibiting the proliferation of breast cancer tissue in a subject which comprises administering to the subject a therapeutically effective amount of a nucleotide sequence encoding a ribozyme, which has the ability to change the transcription/translation of at least one gene selected from the group consisting of those genes identified in Tables 1, 2, 3 or 4.

67. A method for inhibiting the proliferation of breast cancer tissue in a subject which comprises administering to the subject a therapeutically effective amount of a nucleotide sequence encoding a ribozyme, which has the ability to change the transcription/translation of at least one gene selected from the group consisting of those genes identified in Tables 1, 2, 3 or 4.

68. A method for inhibiting the proliferation of breast cancer tissue in a subject which comprises administering to the subject a therapeutically effective amount of a double-stranded RNA corresponding to at least one gene selected from the group consisting of those genes identified in Tables 1, 2, 3 or 4, which has the ability to change the transcription/translation of the at least one gene.

69. A method for inhibiting the proliferation of breast cancer tissue in a subject which comprises administering to the subject a therapeutically effective amount of a double-stranded RNA corresponding to at least one gene selected from the group consisting of those genes identified in Tables 1, 2, 3 or 4, which has the ability to change the transcription/translation of the at least one gene.

70. A method for inhibiting the proliferation of breast cancer tissue in a subject which comprises administering to the subject a therapeutically effective amount of an antagonist that inhibits/activates a protein encoded by at least one gene selected from the group consisting of those genes identified in Tables 1, 2, 3 or 4.

71. The method of claim 70, wherein the antagonist is an antibody specific for the protein.

72. The method of claim 71, wherein the antibody is a monoclonal antibody.

73. The method of claim 72, wherein the monoclonal antibody is conjugated to a toxic reagent.

74. A method for inhibiting the proliferation of breast cancer tissue in a subject which comprises administering to the subject a therapeutically effective amount of an antagonist that inhibits a protein encoded by at least one gene selected from the group consisting of those genes identified in Tables 1, 2, 3 or 4.

75. The method of claim 74, wherein the antagonist is an antibody specific for the protein.

76. The method of claim 75, wherein the antibody is a monoclonal antibody.

77. The method of claim 76, wherein the monoclonal antibody is conjugated to a toxic reagent.

78. A viral vector comprising; a promoter of at least one gene selected from the gene selected from the group consisting of those genes identified in Tables 1, 2, 3 or 4, operably linked to a coding region of a gene that is essential for replication of the vector, wherein the vector is adapted to replicate upon transfection into a breast cell.

79. The vector of claim 78, wherein the viral vector is an adenoviral vector.

80. The vector of claim 78, wherein the coding region of the gene essential for replication of the vector is selected from the group consisting of El a, El b, E2 and E4 coding regions.

81. The vector of claims 78, 79 or 80, further comprising a nucleotide sequence encoding a heterologous gene product.
